SEQUENCE LISTING (20 sequences)

SEQ ID NO: 1 - DNA, 1260 bp, Staphylococcus aureus N315 femB gene sequence
atgaaatttacagagttaactgttaccgaatttgacaactttgtacaaaatccatcattggaaagtcattatttccaagtaaaagaaaatatagttacccgtgagaatgatggctttgaagtagttttattaggtattaaagacgacaataacaaagtaattgcagcaagccttttctctaaaattcctactatgggaagttatgtttactattcgaatcgtggtccagtaatggatttttcagatttaggattagttgattattatttaaaagagttagataaatatttacagcaacatcaatgtttatatgttaaattagatccgtattggttatatcatctatatgataaagatatcgtgccatttgaaggtcgcgagaaaaatgatgccttagtaaacttgtttaaatcacatggttacgagcatcatggctttacaactgagtatgatacatcgagccaagtacgatggatgggcgtattaaaccttgaaggtaaaacacccgaaacattgaaaaagacatttgatagtcaacgtaaacgtaatattaataaagcgataaactatggtgttaaagtcagattccttgaacgtgatgagttcaatcttttcttagatttatatcgtgaaactgaagagcgtgctggatttgtgtcaaaaacagatgattatttttataactttattgacacatatggagataaagtattagtaccattagcatatattgaccttgatgaatatgtgttaaagttgcaacaggaattgaatgacaaagaaaatcgtcgtgatcaaatgatggcgaaagaaaacaaatcagataaacaaatgaagaaaattgcagaattagataagcaaattgatcatgatcagcatgaattattgaatgcaagtgaattgagcaaaacggacggcccaattctaaaccttgcttctggcgtttattttgcaaatgcatatgaagtgaattatttctctggtggttcatcagaaaaatataatcaatttatgggaccatacatgatgcattggtttatgattaactattgcttcgataatggctatgatcgttataatttctatggtttatcaggtgattttacggaaaacagtgaagattatggcgtataccgctttaaacgtggatttaatgtacaaatcgaagaattaataggggatttctataaaccaattcataaagtgaaatattggttgttcacaacattggataaattacgtaaaaaattaaagaaatag

SEQ ID NO: 2 - DNA, 662 bp, Staphylococcus aureus St-au-B01
ctaaaaaagttaaagacagaattttttaggaagggaattatcaaaacatgaaatttacagagttaactgttaccgaatttgacaactttgtacaaaatccatcattggaaagtcattatttccaagtaaaagaaaatatagttacccgtgagaatgatggctttgaagtagttttattaggtattaaagacgacaataacaaagtaattgcagcaagccttttctctaaaattcctactatgggaagttatgtttactattcgaatcgtggtccagtaatggatttttcagatttaggattagttgattattatttaaaagagttagataaatatttacagcaacatcaatgtttatatgttaaattagatccgtattggttatatcatctatatgataaagatatcgtgccatttgaaggtcgcgagaaaaatgatgccttagtaaacttgtttaaatcacatggttacgagcatcatggctttacaactgagtatgatacatcgagccaagtacgatggatgggcgtattaaaccttgaaggtaaaacacccgaaacattgaaaaagacatttgatagtcaacgtaaacgtaatattaataaagcgataaactatggtgttaaagtcagattccttgaacgtgatgagttcaatcttttc

SEQ ID NO: 3 - DNA, 661 bp, Staphylococcus aureus St-au-B02
aaaaagtaaagacagaattttttaggaagggaattatcaaaacatgaaatttacagagttaactgttaccgaatttgacaactttgtacaaaatccatcattggaaagtcattatttccaagtaaaagaaaatatagttacccgtgagaatgatggctttgaagtagttttattaggtattaaagacgacaataacaaagtaattgcagcaagccttttctctaaaattcctactatgggaagttatgtttactattcgaatcgtggtccagtaatggatttttcagatttaggattagttgattattatttaaaagagttagataaatatttacagcaacatcaatgtttatatgttaaattagatccgtattggttatatcatctatatgataaagatatcgtgccatttgaaggtcgcgagaaaaatgatgccttagtaaacttgtttaaatcacatggttacgagcatcatggctttacaactgagtatgatacatcgagccaagtacgatggatgggcgtattaaaccttgaaggtaaaacacccgaaacattgaaaaagacatttgatagtcaacgtaaacgtaatattaataaagcgataaactatggtgttaaagtcagattccttgaacgtgatgagttcaatcttttctta

SEQ ID NO: 4 - DNA, 655 bp, Staphylococcus aureus St-au-B05
gtaaagacagaattttttaggaagggaattatcaaaacatgaaatttacagagttaactgttaccgaatttgacaactttgtacaaaatccatcattggaaagtcattatttccaagtaaaagagaatatagttacccgtgagaatgatggctttgaagtagttttattaggtattaaagacgacaataacaaagtaattgcagcaagccttttctctaaaattcctactatgggaagttatgtttactattcgaatcgtggtccagtaatggatttttcagatttaggattagttgattattatttaaaagagttagataaatatttacagcaacatcaatgtttatatgttaaattagatccgtattggttatatcatctatatgataaagatatcgtgccatttgaaggtcgcgagaaaaatgatgccctagtaaacttgtttaaatcacatggttacgagcatcatggctttacaactgagtatgatacatcgagccaagtacgatggatgggcgtattaaaccttgaaggtaaaacacccgaaacattgaaaaagacatttgatagtcaacgtaaacgtaatattaataaagcgataaactatggtgttaaagtcagattccttgaacgtgatgagttcaatcttttctt

SEQ ID NO: 5 - DNA, 660 bp, Staphylococcus aureus St-au-B06
aaaaagtaaagacagaattttttaggaagggaattatcaaaacatgaaatttacagagttaactgttaccgaatttgacaactttgtacaaaatccatcattggaaagtcattatttccaagtaaaagaaaatatagttacccgtgagaatgatggctttgaagtagttttattaggtattaaagacgacaataacaaagtaattgcagcaagccttttctctaaaattcctactatgggaagttatgtttactattcgaatcgtggtccagtaatggatttttcagatttaggattagttgattattatttaaaagagttagataaatatttacagcaacatcaatgtttatatgttaaattagatccgtattggttatatcatctatatgataaagatatcgtgccatttgaaggtcgcgagaaaaatgatgccttagtaaacttgtttaaatcacatggttacgagcatcatggctttacaactgagtatgatacatcgagccaagtacgatggatgggcgtattaaaccttgaaggtaaaacacccgaaacattgaaaaagacatttgatagtcaacgtaaacgtaatattaataaagcgataaactatggtgttaaagtcagattccttgaacgtgatgagttcaatcttttctt

SEQ ID NO: 6 - DNA, 658 bp, Staphylococcus aureus St-au-B07
aaaaagtaaagacagaattttttaggaagggaattatcaaaacatgaaatttacagagttaactgttaccgaatttgacaactttgtacaaaatccatcattggaaagtcattatttccaagtaaaagaaaatatagttacccgtgagaatgatggctttgaagtagttttattaggtattaaagacgacaataacaaagtaattgcagcaagccttttctctaaaattcctactatgggaagttatgtttactattcgaatcgtggtccagtaatggatttttcagatttaggattagttgattattatttaaaagagttagataaatatttacagcaacatcaatgtttatatgttaaattagatccgtattggttatatcatctatatgataaagatatcgtgccatttgaaggtcgcgagaaaaatgatgccttagtaaacttgtttaaatcacatggttacgagcatcatggctttacaactgagtatgatacatcgagccaagtacgatggatgggcgtattaaaccttgaaggtaaaacacccgaaacattgaaaaagacatttgatagtcaacgtaaacgtaatattaataaagcgataaactatggtgttaaagtcagattccttgaacgtgatgagttcaatcttttc

SEQ ID NO: 7 - DNA, 656 bp, Staphylococcus aureus St-au-B09
gtaaagacagaattttttaggaagggaattatcaaaacatgaaatttacagagttaactgttaccgaatttgacaactttgtacaaaatccatcattggaaagtcattatttccaagtaaaagaaaatatagttacccgtgagaatgatggctttgaagtagttttattaggtattaaagacgacaataacaaagtaattgcagcaagccttttctctaaaattcctactatgggaagttatgtttactattcgaatcgtggtccagtaatggatttttcagatttaggtttagttgattattatttaaaagagttagataaatatttacagcaacatcaatgtttatatgttaaattagatccgtattggttatatcatctatatgataaagatatcgtgccatttgaaggtcgcgagaaaaatgatgccttagtaaacttgtttaaatcgcatggttacgagcatcatggctttacaactgagtatgatacatcgagccaagtacgatggatgggcgtattaaaccttgaaggtaaaacacctgaaacattgaaaaagacatttgatagtcaacgtaaacgtaatattaataaagcgataaactatggtgttaaagtcagattcctcgaacgtgatgagttcaatcttttccta

SEQ ID NO: 8 - DNA, 660 bp, Staphylococcus aureus St-au-B10
aaaagtaaagacagaattttttaggaagggaattatcaaaacatgaaatttacagagttaactgttaccgaatttgacaactttgtacaaaatccatcattggaaagtcattatttccaagtaaaagaaaatatagttacccgtgagaatgatggctttgaagtagttttattaggtattaaagacgacaataacaaagtaattgcagcaagccttttctctaaaattcctactatgggaagttatgtttactattcgaatcgtggtccagtaatggatttttcagatttaggattagttgattattatttaaaagagttagataaatatttacagcaacatcaatgtttatatgttaaattagatccgtattggttatatcatctatatgataaagatatcgtgccatttgaaggtcgcgagaaaaatgatgccttagtaaacttgtttaaatcgcatggttacgagcatcatggctttacaactgagtatgatacatcgagccaagtacgatggatgggcgtattaaaccttgaaggtaaaacacctgaaacattgaaaaagacatttgatagtcaacgtaaacgtaatattaataaagcgataaactatggtgttaaagtcagattccttgaacgtgatgagttcaatcttttccta

SEQ ID NO: 9 - DNA, 658 bp, Staphylococcus aureus St-au-B11
aagttaaagacagaattttttaggaagggaattatcaaaacatgaaatttacagagttaactgttaccgaatttgacaactttgtacaaaatccatcattggaaagtcattatttccaagtaaaagaaaatatagttacccgtgagaatgatggctttgaagtagttttattaggtattaaagacgacaataacaaagtaattgcagcaagccttttctctaaaattcctactatgggaagttatgtttactattcgaatcgtggtccagtaatggatttttcagatttaggattagttgattattatttaaaagagttagataaatatttacagcaacatcaatgtttatatgttaaattagatccgtattggttatatcatctatatgataaagatatcgtgccatttgaaggtcgcgagaaaaatgatgccttagtaaacttgtttaaatcgcatggttacgagcatcatggctttacaactgagtatgatacatcgagccaagtacgatggatgggcgtattaaaccttgaaggtaaaacacctgaaacattgaaaaagacatttgatagtcaacgtaaacgtaatattaataaagcgataaactatggtgttaaagtcagattccttgaacgtgatgagttcaatcttttcct

SEQ ID NO: 10 - DNA, 628 bp, Staphylococcus aureus St-au-B12
tttttaggaagggaattatcaaaacatgaaatttacagagttaactgttaccgaatttgacaactttgtacaaaatccatcattggaaagtcattatttccaagtaaaagaaaatatagttacccgtgagaatgatggctttgaagtagttttattaggtattaaagacgacaataacaaagtaattgcagcaagccttttctctaaaattcctactatgggaagttatgtttactattcgaatcgtggtccagtaatggatttttcagatttaggattagttgattattatttaaaagagttagataaatatttacagcaacatcaatgtttatatgttaaattagatccgtattggttatatcatctatatgataaagatatcgtgccatttgaaggtcgcgagaaaaatgatgccttagtaaacttgtttaaatcacatggttacgagcatcatggctttacaactgagtatgatacatcgagccaagtacgatggatgggcgtattaaaccttgaaggtaaaacacccgaaacattgaaaaagacatttgatagtcaacgtaaacgtaatattaataaagcgataaactatggtgttaaagtcagattccttgaacgtgatgag

SEQ ID NO: 11 - DNA, 662 bp, Staphylococcus aureus St-au-B14
aaaaaagtaaagacagaattttttaggaagggaattatcaaaacatgaaatttacagagttaactgttaccgaatttgacaactttgtacaaaatccatcattggaaagtcattatttccaagtaaaagaaaatatagttacccgtgagaatgatggctttgaagtagttttattaggtattaaagacgacaataacaaagtaattgcagcaagccttttctctaaaattcctactatgggaagttatgtttactattcgaatcgtggtccagtaatggatttttcagatttaggattagttgattattatttaaaagagttagataaatatttacagcaacatcaatgtttatatgttaaattagatccgtattggttatatcatctatatgataaagatatcgtgccatttgaaggtcgcgagaaaaatgatgccttagtaaacttgtttaaatcacatggttacgagcatcatggctttacaactgagtatgatacatcgagccaagtacgatggatgggcgtattaaaccttgaaggtaaaacacccgaaacattgaaaaagacatttgatagtcaacgtaaacgtaatattaataaagcgataaactatggtgttaaagtcagattccttgaacgtgatgagttcaatcttttctta

SEQ ID NO: 12 - DNA, 85 bp, Staphylococcus aureus
  femB consensus sequence
crcatggttacgagcatcatggctttacaactgagtatgatacatcgagccaagtacgatggatgggcgtattaaaccttgaagg

SEQ ID NO: 13 - DNA, 24 bp, Staphylococcus aureus
  femB highly conserved sequence
tgagtatgatacatcgagccaagt

SEQ ID NO: 14 - DNA, 18 bp, Artificial Sequence
  Forward Primer sequence
cacatggttacgagcatc

SEQ ID NO: 15 - DNA, 19 bp, Artificial Sequence
  Reverse Primer sequence
ccttcaaggtttaatacgc

SEQ ID NO: 16 - DNA, 36 bp, Artificial Sequence
  Molecular Beacon sequence with stem
cgcgcatgagtatgatacatcgagccaagttgcgcg

SEQ ID NO: 17 - DNA, 24 bp, Artificial Sequence
  Molecular Beacon loop sequence
tgagtatgatacatcgagccaagt

SEQ ID NO: 18 - DNA, 36 bp, Artificial Sequence
  Molecular Beacon sequence with stem
cgcgcaacttggctcgatgtatcatactcatgcgcg

SEQ ID NO: 19 - DNA, 24 bp, Artificial Sequence
  Molecular Beacon loop sequence
acttggctcgatgtatcatactca

SEQ ID NO: 20 - DNA, 18 bp, Artificial Sequence
  Forward Primer sequence
crcatggttacgagcatc

(19) United States
(12) Patent Application Publication
(10) Pub. No.: US 2008/0108060 A1
(43) Pub. Date: May 8, 2008

(54) Polynucleotides for the Detection of Staphylococcus Aureus

(75) Inventors: Eliane Ubalijoro, Québec (CA); Daniel Plante, Quebec (CA); Alexandre Hebert, Quebec (CA)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: WARNEX RESEARCH INC., Laval, QC (CA)

(21) Appl. No.: 11/579,306

(22) PCT Filed: May 10, 2005
(86) PCT No.: PCT/CA05/00710

§ 371 Date: Jul. 23, 2007

Related U.S. Application Data
(60) Provisional application No. 60/569,762, filed on May 10, 2004.

(51) Int. Cl. C07H 21/04; C12Q 1/68

(52) U.S. Cl. 435/6; 536/24.33

(57) ABSTRACT

Polynucleotide primers and probes for the specific amplification and detection of S. aureus in samples are provided. The polynucleotide primers and probes are targeted to the S. aureus gene and are capable of detecting a wide range of S. aureus strains. The primers and probes can be used in real time diagnostic assays for rapid detection of S. aureus in a variety of situations. Kits comprising the primers and probes are also provided.

### FIELD OF THE INVENTION

The present invention pertains to the field of detection of microbial contaminants. More specifically, the invention relates to the detection of contamination by Staphylococcus aureus.

### BACKGROUND OF THE INVENTION

Staphylococcus aureus (S. aureus) accounts for an estimated 14% of all foodborne disease outbreaks in the United States. This bacterium is commonly associated with foods such as meat, meat products, poultry, eggs, bakery products, and foods that require handling during preparation and that are kept at slightly elevated temperatures. S. aureus produces heat stable toxins and ingestion of foods contaminated with these toxins cause illnesses in humans. Within 2 to 4 hours after ingestion of foods contaminated with toxin, individuals may develop symptoms including nausea, vomiting, abdominal cramps and retching. In severe cases, symptoms may include headache, muscle cramping, and changes in pulse rate and blood pressure. S. aureus infections are also responsible for other diseases, such as Toxic Shock Syndrome and skin infections, which are usually caused by the colonization of the body by S. aureus (Holmberg, S D (1984) Journal of the American Medical Association 251:487-489).

In order to prevent the occurrence of S. aureus infections, methods of detection can be employed that identify the presence of the bacteria in food prior to consumer availability and consumption. Many detection techniques, however, require long time periods and, therefore, are not time and cost effective due to relatively quick rates of food spoilage. For example, a number of detection technologies require the culturing of bacterial samples for time periods of up to eight days. During that time, however, the product being tested must be placed in circulation for purchase and consumption. Therefore, a system that can rapidly identify the presence of S. aureus in food and other test samples is desirable.

A variety of methods have been described for the detection of bacterial contaminants. One of these methods is the amplification of specific nucleotide sequences using specific primers in a PCR assay. Upon completion of the amplification of a target sequence, the presence of an amplicon is detected using agarose gel electrophoresis.

For example, a multiplex PCR-based method of simultaneously identifying S. aureus and detecting methicillin and mupirocin resistance in isolates has been described (Perez-Roth, E. et al., (2001) Journal of Clinical Microbiology. 39:4037-4041). This method involves a triplex PCR amplification of a 651 base pair fragment of the femB gene, a 456 base pair fragment of the ileS-2 gene and a 310 base pair fragment of the mecA gene and the detection of the amplified fragments on ethidium bromide-stained agarose gels. The PCR protocol described in this method, however, is not specific for the detection of S. aureus, since the femB primers utilised in the protocol are capable of amplifying the corresponding region of the femB gene from Staphylococcus auricularis. In addition, multiplex PCR may require a considerable amount of optimisation in order to determine the optimal conditions that permit all primers to bind effectively to and amplify their target sequences.

Furthermore, detection using agarose gel electrophoresis, while being more rapid than traditional methods requiring culturing bacterial samples, is still relatively time consuming and subject to post-PCR contamination during the running of the agarose gel.

Nucleic acid hybridization is another technology often utilized in conjunction with PCR for detection of bacterial contamination. In such detection methodologies, the target sequence of interest is amplified and then hybridized to an oligonucleotide probe which possesses a complementary nucleic acid sequence to that of the target molecule. The probe is modified so that detection of the hybridization product can occur, for example, the probe can be labelled with a radioisotope or fluorescent moiety.

A particularly useful modification of the above technology provides for the concurrent amplification and detection of the target sequence (i.e. in “real time”) through the use of specially adapted oligonucleotide probes. Examples of such probes include molecular beacon probes (Tyagi et al., (1996) Nature Biotechnol. 14:303-308), TaqMan® probes (U.S. Pat. Nos. 5,691,146 and 5,876,930) and Scorpion probes (Whitcombe et al., (1999) Nature Biotechnol. 17:804-807).

Molecular beacons represent a powerful tool for the rapid detection of specific nucleotide sequences and are capable of detecting the presence of a complementary nucleotide sequence even in homogenous solutions. Molecular beacons can be described as hairpin stem-and-loop oligonucleotide sequences, in which the loop portion of the molecule represents a probe sequence, which is complementary to a predetermined sequence in a target nucleotide. One arm of the beacon sequence is attached to a fluorescent moiety, while the other arm of the beacon is attached to a non-fluorescent quencher. The stem portion of the stem-and-loop sequence holds the two arms of the beacon in close proximity. Under these circumstances, the fluorescent moiety is quenched. When the beacon encounters a nucleic acid sequence complementary to its probe sequence, the probe hybridizes to the nucleic acid sequence, forming a stable complex and, as a result, the arms of the probe are separated and the fluorophore emits light. Thus, the emission of light is indicative of the presence of the specific nucleic acid sequence. Individual molecular beacons are highly specific for the DNA sequences they are complementary to.

U.S. Pat. No. 6,468,743 describes an assay based on PCR techniques for detecting microbial contaminants in foodstuffs that utilises primers and molecular beacon probes. The assay relies on detection of either a universal bacterial or viral sequence, or a sequence that is specific to a microorganism or virus. For example, the entA, entB, entC1, entD, entE, tst, eat, etb and nuc genes are identified as potential targets for detection of S. aureus.

The FemB protein is an enzyme involved in the synthesis of macromolecules that form the staphylococcal cell wall [Stapleton, P D and Taylor P W (2002) Science Progress 85:57-72; Stranden, A M et al. (1997) Journal of Bacteriology 179:9-16].

The sequences of various fragments of the S. aureus genome that include an open reading frame corresponding to the femB gene are described in U.S. Pat. No. 6,593,114. International Patent Application No. PCT/IB02/02637 (WO 02/094868) describes the identification of 2821 nucleic acid coding sequences from S. aureus, including the femB gene, and the proteins that they encode. The nucleic acid sequences are described as being useful for the development of vaccines, for diagnosis and detection of S. aureus infections and as potential targets for the development of anti-bacterial drugs. The femB gene was also identified as a S. aureus virulence-associated gene that could serve as a target for the development of new anti-bacterial agents, or that could be used to develop novel S. aureus mutants useful as vaccines (see U.S. Pat. Nos. 6,485,899, and 6,455,323).

Detection of S. aureus utilising TaqMan® probes which are described as being targeted to the genes encoding staphylococcal enterotoxins A to D, the mecA gene (responsible for methicillin resistance) and the femB gene has been reported (Klotz, M. et al., (2003) Journal of Clinical Microbiology. 41:4683-4687). However, the primers and probe described in this publication as being targeted to the femB gene are, in fact, complementary to regions of another S. aureus gene: fmhB (Accession No. AF106850; Tschierske, M. et al., (1999) FEMS Microbiol. Lett. 171:97-102), rather than to femB.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide polynucleotides for the detection of S. aureus. In accordance with one aspect of the present invention, there is provided a combination of polynucleotides for amplification and detection of a S. aureus target nucleotide sequence, said combination comprising: (a) a first polynucleotide primer comprising at least 7 consecutive nucleotides of the sequence as set forth in SEQ ID NO:1; (b) a second polynucleotide primer comprising at least 7 consecutive nucleotides of a sequence complementary to SEQ ID NO:1; and (c) a polynucleotide probe comprising at least 7 consecutive nucleotides of the sequence as set forth in SEQ ID NO:12, or the complement thereof.

In accordance with another aspect of the invention, there is provided a method of detecting S. aureus in a sample, said method comprising: (i) contacting a sample suspected of containing, or known to contain, a S. aureus target nucleotide sequence with a combination of polynucleotide primers capable of amplifying said target nucleotide sequence under conditions that permit amplification of said target nucleotide sequence, said polynucleotide primers comprising: (a) a first polynucleotide primer comprising at least 7 consecutive nucleotides of the sequence as set forth in SEQ ID NO:1; and (b) a second polynucleotide primer comprising at least 7 consecutive nucleotides of a sequence complementary to SEQ ID NO:1, and said target nucleotide sequence being a portion of a S. aureus femB gene of less than about 500 nucleotides in length and comprising at least 55 consecutive nucleotides of the sequence set forth in SEQ ID NO:12, and (ii) detecting any amplified target nucleotide sequence, wherein detection of an amplified target nucleotide sequence indicates the presence of S. aureus in the sample.

In accordance with another aspect of the invention, there is provided a method of detecting S. aureus in a sample, said method comprising the steps of: (i) contacting a sample suspected of containing, or known to contain, a S. aureus target nucleotide sequence with a combination of polynucleotides of the invention under conditions that permit amplification of said target nucleotide sequence, and (ii) detecting any amplified target nucleotide sequence, wherein detection of an amplified target nucleotide sequence indicates the presence of S. aureus in the sample.

In accordance with another aspect of the invention, there is provided a kit for the detection of S. aureus in a sample, said kit comprising: (a) a first polynucleotide primer comprising at least 7 consecutive nucleotides of the sequence as set forth in SEQ ID NO:1; (b) a second polynucleotide primer comprising at least 7 consecutive nucleotides of a sequence complementary to SEQ ID NO:1; and (c) a polynucleotide probe comprising at least 7 consecutive nucleotides of the sequence as set forth in SEQ ID NO:12, or the complement thereof.

In accordance with another aspect of the invention, there is provided a pair of polynucleotide primers for amplification of a portion of a S. aureus femB gene sequence, said portion being less than about 500 nucleotides in length and comprising at least 55 consecutive nucleotides of the sequence set forth in SEQ ID NO:12, said pair of polynucleotide primers comprising: (a) a first polynucleotide primer comprising at least 7 consecutive nucleotides of the sequence as set forth in SEQ ID NO:1; and (b) a second polynucleotide primer comprising at least 7 consecutive nucleotides of a sequence complementary to SEQ ID NO:1.

In accordance with another aspect of the invention, there is provided an isolated S. aureus specific polynucleotide consisting essentially of: (a) the sequence as set forth in SEQ ID NO:12, or a fragment of said sequence, or (b) a sequence that is the complement of (a).

In accordance with another aspect of the invention, there is provided a polynucleotide primer of between 7 and 100 nucleotides in length for amplification of a portion of a S. aureus femB gene sequence, said polynucleotide primer comprising at least 7 consecutive nucleotides of the sequence as set forth in SEQ ID NO:12, or the complement thereof.

In accordance with another aspect of the invention, there is provided a polynucleotide probe of between 7 and 100 nucleotides in length for detection of S. aureus nucleic acids, said polynucleotide probe comprising at least 7 consecutive nucleotides of the sequence as set forth in SEQ ID NO:12, or the complement thereof.

### BRIEF DESCRIPTION OF THE DRAWINGS

These and other features of the invention will become more apparent in the following detailed description in which reference is made to the appended drawings wherein:

FIG. 1 presents a multiple alignment showing conserved regions of a portion of the femB gene from several S. aureus strains [SEQ ID NOs:2-11]. Shaded blocks highlight the following regions: bases 464 to 481: forward primer SEQ ID NO:14; bases 495 to 518: binding site for molecular beacon probe #1 [SEQ ID NO:16]; bases 530 to 548: binding site for reverse primer SEQ ID NO:15;

FIG. 2 presents the arrangement of PCR primers and a molecular beacon probe on the femB gene sequence in one embodiment of the invention. Numbers in parentheses indicate the positions of the first and last nucleotides of each feature on the PCR product generated with primers SEQ ID NOs:14 and 15;

FIG. 3 presents the secondary structure of a molecular beacon probe [SEQ ID NO:16] in accordance with one embodiment of the invention; and

FIG. 4 presents the sequence of (A) a S. aureus femB gene [SEQ ID NO:1]; (B) a conserved region of the S. aureus femB gene, which is unique to S. aureus isolates [SEQ ID NO:12], and (C) a 24 nucleotide sequence found within the conserved region, which is exclusive to S. aureus isolates [SEQ ID NO:13].

### DETAILED DESCRIPTION OF THE INVENTION

The present invention is based on the identification of a highly conserved region (consensus sequence) that is common to strains of S. aureus. The consensus sequence constitutes a suitable target sequence for the design of primers and probes capable of specifically amplifying and detecting S. aureus in a test sample.

The present invention provides for primer and probe sequences capable of amplifying and/or detecting all or part of the consensus sequence that are suitable for use in detecting the presence of S. aureus bacteria in a range of samples including, but not limited to, clinical samples, microbiological pure cultures, food, and environmental and pharmaceutical quality control processes. In one embodiment, the invention provides diagnostic assays that can be carried out in real time and addresses the need for rapid detection of S. aureus in a variety of biological samples.

The primers and probes of the present invention are capable of specifically detecting S. aureus even in the presence of nucleic acids from other non-S. aureus nucleic acids. In accordance with the present invention, the primers and probes demonstrate a specificity for S. aureus nucleic acid sequences of at least 95%, as defined herein. In one embodiment, the primers and probes of the invention demonstrate a specificity for S. aureus nucleic acid sequences of at least 97%. In another embodiment, the primers and probes demonstrate a specificity for S. aureus nucleic acid sequences of at least 98%. In further embodiments, the primers and probes demonstrate a specificity for S. aureus nucleic acid sequences of at least 99%, and at least 99.5%.

In addition, the primers and probes of the present invention are capable of specifically detecting S. aureus target sequences from a range of S. aureus strains. In accordance with the present invention, the primers and probes demonstrate a sensitivity of at least 95%, as defined herein. In one embodiment, the primers and probes of the invention demonstrate a sensitivity of at least 97%. In another embodiment, the primers and probes demonstrate a sensitivity of at least 98%. In further embodiments, the primers and probes demonstrate a sensitivity of at least 99%, and at least 99.5%.

### Definitions

Unless defined otherwise, all technical and scientific terms used herein have the same meaning as commonly understood by one of ordinary skill in the art to which this invention belongs.

The term “polynucleotide,” as used herein, refers to a polymer of greater than one nucleotide in length of ribonucleic acid (RNA), deoxyribonucleic acid (DNA), hybrid RNA/DNA, modified RNA or DNA, or RNA or DNA mimetics. The polynucleotides may be single- or double-stranded. The term includes polynucleotides composed of naturally-occurring nucleobases, sugars and covalent internucleoside (backbone) linkages as well as polynucleotides having non-naturally-occurring portions which function similarly. Such modified or substituted polynucleotides are well-known in the art and for the purposes of the present invention, are referred to as “analogues.”

The terms “primer” and “polynucleotide primer,” as used herein, refer to a short, single-stranded polynucleotide capable of hybridizing to a complementary sequence in a nucleic acid sample. A primer serves as an initiation point for template-dependent nucleic acid synthesis. Nucleotides are added to a primer by a nucleic acid polymerase, which adds such nucleotides in accordance with the sequence of the template nucleic acid strand. A “primer pair” or “primer set” refers to a set of primers including a 5′ upstream primer that hybridizes with the 5′ end of the sequence to be amplified and a 3′ downstream primer that hybridizes with the complementary 3′ end of the sequence to be amplified. The term “forward primer” as used herein, refers to a primer which anneals to the 5′ end of the sequence to be amplified. The term “reverse primer”, as used herein, refers to a primer which anneals to the complementary 3′ end of the sequence to be amplified.

The terms “probe” and “polynucleotide probe,” as used herein, refer to a polynucleotide used for detecting the presence of a specific nucleotide sequence in a sample. Probes specifically hybridize to a target nucleotide sequence, or the complementary sequence thereof, and may be single- or double-stranded.

The term “specifically hybridize,” as used herein, refers to the ability of a polynucleotide to bind detectably and specifically to a target nucleotide sequence. Polynucleotides, oligonucleotides and fragments thereof specifically hybridize to target nucleotide sequences under hybridization and wash conditions that minimize appreciable amounts of detectable binding to non-specific nucleic acids. High stringency conditions can be used to achieve specific hybridization conditions as is known in the art. Typically, hybridization and washing are performed at high stringency according to conventional hybridization procedures and employing one or more washing step in a solution comprising 1-3×SSC, 0.1-1% SDS at 50-70° C. for 5-30 minutes.

The term “specificity,” as used herein, refers to the ability of a primer or primer pair to amplify, or a probe to detect, nucleic acid sequences from S. aureus but not other bacterial species. “% specificity” is defined by a negative validation test as described herein whereby the primers and/or probe are tested against a panel of at least 100 bacterial species other than S. aureus. Thus, for example, a pair of primers that does not amplify any nucleic acid sequences from the panel of bacterial species would be defined as demonstrating 100% specificity and a pair of primers that amplified a nucleic acid sequence from one bacterial species in a panel of 100 species would be defined as demonstrating 99% specificity.

The term “sensitivity,” as used herein, refers to the ability of a primer or primer pair to amplify, or a probe to detect, nucleic acid sequences from a range of S. aureus strains. “% sensitivity” is defined by a positive validation test as described herein whereby the primers and/or probe are tested against a panel of at least 20 S. aureus strains. Thus, for example, a pair of primers that amplifies nucleic acid sequences from all S. aureus strains in the panel would be defined as demonstrating 100% sensitivity and a pair of primers that amplified nucleic acid sequences from nineteen S. aureus strains in a panel of 20 strains would be defined as demonstrating 95% sensitivity.

The term “corresponding to” refers to a polynucleotide sequence that is identical to all or a portion of a reference polynucleotide sequence. In contradistinction, the term “complementary to” is used herein to indicate that the a polynucleotide sequence is identical to all or a portion of the complementary strand of a reference polynucleotide sequence. For illustration, the nucleotide sequence “TATAC” corresponds to a reference sequence “TATAC” and is complementary to a reference sequence “GTATA.”

The terms “hairpin” or “hairpin loop” refer to a single strand of DNA or RNA, the ends of which comprise complementary sequences, whereby the ends anneal together to form a “stem” and the region between the ends is not annealed and forms a “loop.” Some probes, such as molecular beacons, have such “hairpin” structure when not hybridized to a target sequence. The loop is a single-stranded structure containing sequences complementary to the target sequence, whereas the stem self-hybridises to form a double-stranded region and is typically unrelated to the target sequence. Nucleotides that are both complementary to the target sequence and that can self-hybridise can be included in the stem region.

The terms “target sequence” or “target nucleotide sequence,” as used herein, refer to a particular nucleic acid sequence in a test sample to which a primer and/or probe is intended to specifically hybridize. A “target sequence” is typically longer than the primer or probe sequence and thus can contain multiple “primer target sequences” and “probe target sequences.” A target sequence may be single or double stranded. The term “primer target sequence” as used herein refers to a nucleic acid sequence in a test sample to which a primer is intended to specifically hybridize. The term “probe target sequence” refers to a nucleic acid sequence in a test sample to which a probe is intended to specifically hybridize.

As used herein, the term “about” refers to a +/−10% variation from the nominal value. It is to be understood that such a variation is always included in any given value provided herein, whether or not it is specifically referred to.

### Target Sequence

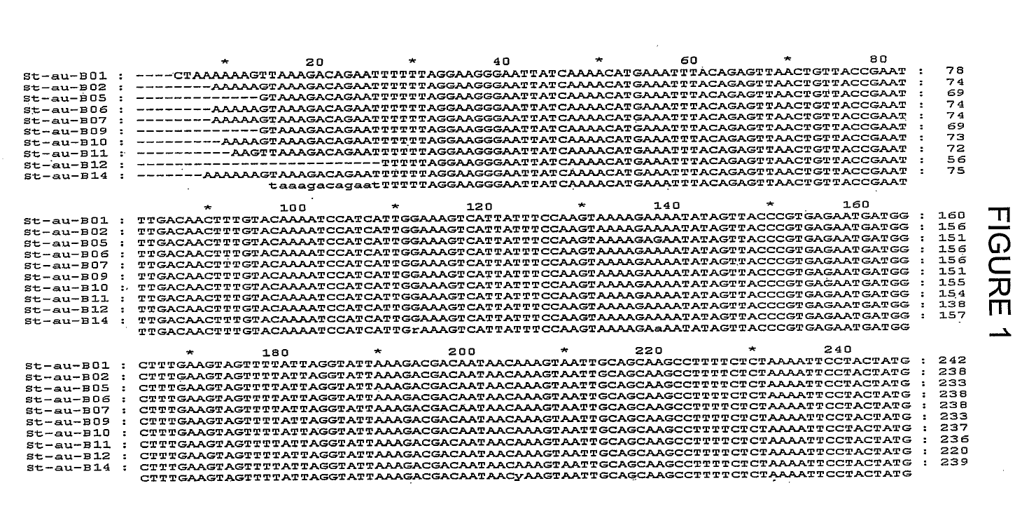

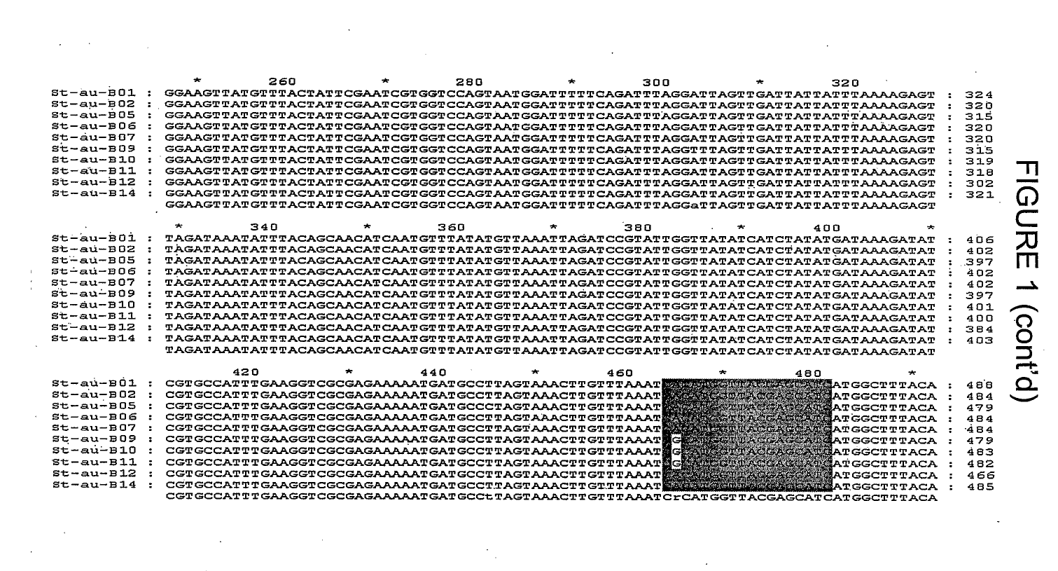

In order to identify highly conserved regions of the femB gene that could potentially serve as target sequences for specific probes, S. aureus femB gene sequences (having a general sequence corresponding to SEQ ID NO:1) were subjected to a multiple alignment analysis. An exemplary multiple sequence alignment of portions of the coding strand of the femB gene from a number of S. aureus strains is shown in FIG. 1. One skilled in the art will appreciate that similar alignments can be conducted using longer or shorter sequences, such as the sequence shown in FIG. 7A [SEQ ID NO:1].

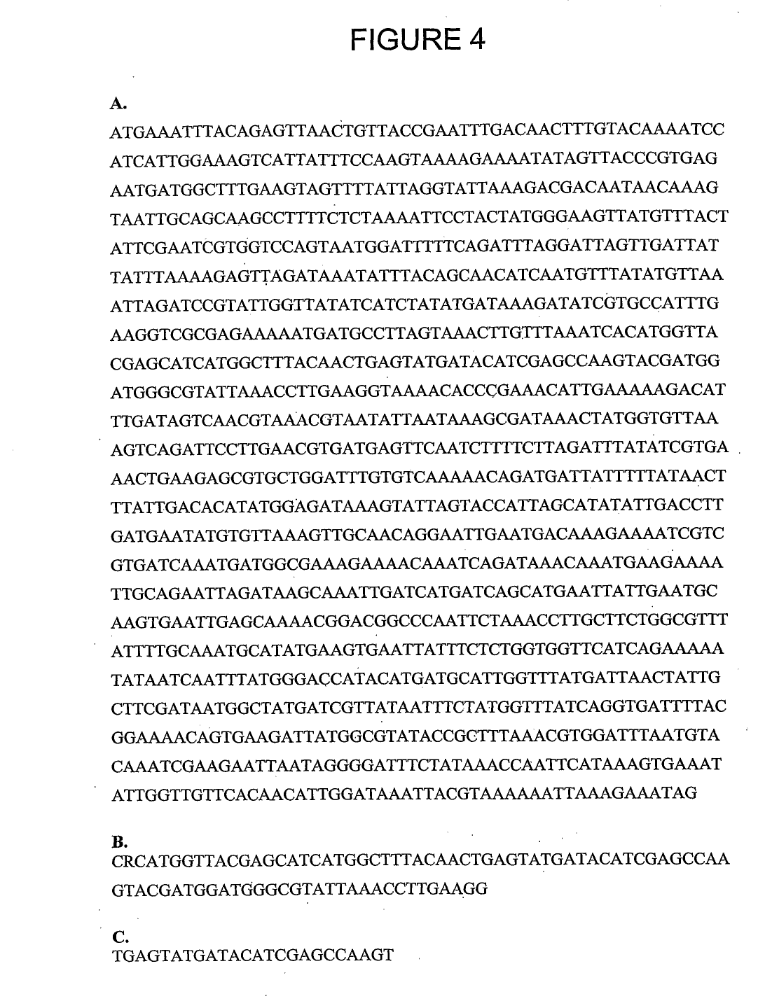

An 85 nucleotide region of the femB gene sequence, having a sequence corresponding to SEQ ID NO:12 (shown below and in FIG. 4B), was identified as being generally conserved in S. aureus isolates. This sequence is referred to herein as a consensus sequence.

[SEQ ID NO:12]

5′-CR*CATGGTTACGAGCATCATGGCTTTACAACTGAGTATGATACATC
GAGCCAAGTACGATGGATGGGCGTATTAAACCTTGAAGG-3′

Accordingly, the present invention provides an isolated S. aureus specific polynucleotide consisting of the consensus sequence as set forth in SEQ ID NO:12, or the complement thereof, that can be used as a target sequence for the design of probes for the specific detection of S. aureus.

It will be recognised by those skilled in the art that all, or a portion, of the consensus sequence set forth in SEQ ID NO:12 can be used as a target sequence for the specific detection of S. aureus. Thus, in one embodiment of the invention, a target sequence suitable for the specific detection of S. aureus that comprises at least 60% of the sequence set forth in SEQ ID NO:12, or the complement thereof, is provided. In another embodiment, the target sequence comprises at least 65% of the sequence set forth in SEQ ID NO:12, or the complement thereof. In a further embodiment, the target sequence comprises at least 75% of the sequence set forth in SEQ ID NO:12, or the complement thereof. Target sequences comprising at least 80%, 85%, 90%, 95% and 98% of the sequence set forth in SEQ ID NO:12, or the complement thereof, are also contemplated.

Such portions of the consensus sequence can also be expressed in terms of consecutive nucleotides of the sequence set forth in SEQ ID NO:12. Accordingly, target sequences comprising portions of the consensus sequence including at least 55, at least 60, at least 65, at least 70, at least 75, and at least 80 consecutive nucleotides of the sequence set forth in SEQ ID NO:12, or the complement thereof, are contemplated. By “at least 55 consecutive nucleotides” it is meant that the target sequence may comprise any number of consecutive nucleotides between 55 and the full-length of the sequence set forth in SEQ ID NO:12, thus this range includes portions of the consensus sequence that comprise at least 56, at least 57, at least 58, at least 59, etc, consecutive nucleotides of the sequence set forth in SEQ ID NO:12, or the complement thereof.

Within the 85 nucleotide consensus sequence, an additional highly conserved 24 nucleotide region, having a sequence corresponding to SEQ ID NO:13, was identified (shown below and in FIG. 4C).

| | |
|---|---|
| 5′-TGAGTATGATACATCGAGCCAAGT-3′ | [SEQ ID NO:13] |

Accordingly, one embodiment of the present invention provides for target sequences that comprise all or a portion of a sequence corresponding to SEQ ID NO:13, or the complement thereof.

It will also be appreciated that the target sequence may include additional nucleotide sequences that are found upstream and/or downstream of the consensus sequence in the S. aureus genome. As the assays provided by the present invention typically include an amplification step, it may be desirable to select an overall length for the target sequence such that the assay can be conducted fairly rapidly. Thus, the target sequence typically has an overall length of less than about 500 nucleotides. In one embodiment, the target sequence has an overall length of less than about 400 nucleotides. In another embodiment, the target sequence has an overall length of less than about 350 nucleotides. In other embodiments, the target sequence has an overall length of less than or equal to about 300, about 250, about 200, about 190, about 180, about 150 and about 100 nucleotides. In a further embodiment, the target sequence has an overall length corresponding to the length of the consensus sequence, i.e. 85 nucleotides.

### Polynucleotide Primers and Probes

The present invention provides for polynucleotides for the amplification and/or detection of S. aureus nucleic acids in a sample. The polynucleotide primers and probes of the invention comprise a sequence that corresponds to or is complementary to a portion of the S. aureus femB gene sequence and are capable of specifically hybridizing to S. aureus nucleic acids. In one embodiment, the polynucleotide primers and probes of the invention comprise a sequence that corresponds to or is complementary to a portion of the S. aureus femB gene sequence as set forth in SEQ ID NO:1. In a further embodiment, the polynucleotide primers and probes comprise a sequence that corresponds to or is complementary to a portion of the S. aureus femB gene sequence as set forth in any one of SEQ ID NOs:2-11 (shown in FIG. 1).

The polynucleotides of the present invention are generally between about 7 and about 100 nucleotides in length. One skilled in the art will understand that the optimal length for a selected polynucleotide will vary depending on its intended application (i.e. primer, probe or combined primer/probe) and on whether any additional features, such as tags, self-complementary “stems” and labels (as described below), are to be incorporated. In one embodiment of the present invention, the polynucleotides are between about 10 and about 100 nucleotides in length. In another embodiment, the polynucleotides are between about 12 and about 100 nucleotides in length. In other embodiments, the polynucleotides are between about 12 and about 50 nucleotides and between about 12 and about 40 nucleotides in length.

One skilled in the art will also understand that the entire length of the polynucleotide primer or probe does not need to correspond to or be complementary to the S. aureus femB gene sequence in order to specifically hybridize thereto. Thus, the polynucleotide primers and probes may comprise nucleotides at the 5′ and/or 3′ termini that are not complementary to the S. aureus femB gene sequence. Such non-complementary nucleotides may provide additional functionality to the primer/probe, for example, they may provide a restriction enzyme recognition sequence or a “tag” that facilitates detection, isolation or purification. Alternatively, the additional nucleotides may provide a self-complementary sequence that allows the primer/probe to adopt a hairpin configuration. Such configurations are necessary for certain probes, for example, molecular beacon and Scorpion probes.

The present invention also contemplates that one or more positions within the polynucleotide can be degenerate, i.e. can be filled by one of two or more alternate nucleotides. As is known in the art, certain positions in a gene can vary in the nucleotide that is present at that position depending on the strain of bacteria that the gene originated from. By way of example, position 465 of the alignment shown in FIG. 1 can contain an adenine (“A”) nucleotide or a guanine (“G”) nucleotide depending on which strain of S. aureus the femB gene originates from. Thus, a “degenerate” primer or probe can be designed to target this sequence that contains either an A or a G at the position corresponding to position 465 in the alignment. Degenerate primers or probes are typically prepared by synthesising a “pool” of polynucleotide primers or probes, for example, a pool that contains approximately equal amounts of a polynucleotide containing an A at the degenerate position and a polynucleotide containing a G at the degenerate position.

Typically, the polynucleotide primers and probes of the invention comprise a sequence of at least 7 consecutive nucleotides that correspond to or are complementary to a portion of the S. aureus femB gene sequence. As is known in the art, the optimal length of the sequence corresponding or complementary to the S. aureus femB gene sequence will be dependent on the specific application for the polynucleotide, for example, whether it is to be used as a primer or a probe and, if the latter, the type of probe. Optimal lengths can be readily determined by the skilled artisan.

In one embodiment, the polynucleotides comprise at least 10 consecutive nucleotides corresponding or complementary to a portion of the S. aureus femB gene sequence. In another embodiment, the polynucleotides comprise at least 12 consecutive nucleotides corresponding or complementary to a portion of the S. aureus femB gene sequence. In a further embodiment, the polynucleotides comprise at least 15 consecutive nucleotides corresponding or complementary to a portion of the S. aureus femB gene sequence. Polynucleotides comprising at least 18, at least 20, at least 22 and at least 24 consecutive nucleotides corresponding or complementary to a portion of the S. aureus femB gene sequence are also contemplated.

Sequences of exemplary polynucleotides of the invention are set forth in Table 1. Further non-limiting examples for the polynucleotides of the invention include polynucleotides that comprise at least 7 consecutive nucleotides of any one of SEQ ID NOs:12, 13, 14, 15, 17, 19 and 20.

TABLE 1
Exemplary polynucleotides of the invention

| | |
|---|---|
| Nucleotide sequence | SEQ ID NO |

| | |
|---|---|
| 5′-CACATGGTTACGAGCATC-3′ | 14 |
| 5′-CCTTCAAGGTTTAATACGC-3′ | 15 |
| 5′-TGAGTATGATACATCGAGCCAAGT-3′ | 17 |
| 5′-ACTTGGCTCGATGTATCATACTCA-3′ | 19 |
| 5′-CR*CATGGTTACGAGCATC-3′ | 20 |

*R represents either A or G.

### Primers

As indicated above, the polynucleotide primers of the present invention comprise a sequence that corresponds to or is complementary to a portion of the S. aureus femB gene sequence. In accordance with the invention, the primers are capable of amplifying a target nucleotide sequence comprising all or a portion of the 85 nucleotide consensus sequence as shown in SEQ ID NO:12. Accordingly, in one embodiment the present invention provides for primer pairs capable of amplifying a S. aureus target nucleotide sequence, wherein the target sequence is less than about 500 nucleotides in length and comprises at least 55 consecutive nucleotides of SEQ ID NO:12, or the complement thereof.

Thus, pairs of primers can be selected to comprise a forward primer corresponding to a portion of the S. aureus femB gene sequence upstream of or within the region of the gene corresponding to SEQ ID NO:12 and a reverse primer that it is complementary to a portion of the S. aureus femB gene sequence downstream of or within the region of the gene corresponding to SEQ ID NO:12. In accordance with the present invention, the primers comprise at least 7 consecutive nucleotides of the sequence set forth in SEQ ID NO:1. In one embodiment, the primers comprise at least 7 consecutive nucleotides of the sequence as set forth in any one of SEQ ID NOs:2-11. In another embodiment, the primers comprise at least 7 consecutive nucleotides of the sequence set forth in SEQ ID NO:12.

Appropriate primer pairs can be readily determined by a worker skilled in the art. In general, primers are selected that specifically hybridize to a portion of the S. aureus femB gene sequence without exhibiting significant hybridization to non-S. aureus femB nucleic acids. In addition, primers are selected that contain minimal sequence repeats and that demonstrate a low potential for forming dimers, cross dimers, or hairpin structures and for cross priming. Such properties can be determined by methods known in the art, for example, using the computer modelling program OLIGO® Primer Analysis Software (distributed by National Biosciences, Inc., Plymouth, Minn.).

Non-limiting examples of suitable primer sequences include sequences that comprise SEQ ID NO:14, 15 or 20 shown in Table 1, as well as primers comprising at least 7 consecutive nucleotides of any one of SEQ ID NOs: 14, 15, 17, 19 or 20.

### Probes

In order to specifically detect S. aureus, the probe polynucleotides of the invention are designed to correspond to or be complementary to a portion of the consensus sequence shown in SEQ ID NO:12. The probe polynucleotides, therefore, comprise at least 7 consecutive nucleotides of the sequence set forth in SEQ ID NO:12, or the complement thereof. As indicated above, a highly conserved 24 nucleotide region was identified within the femB consensus sequence. In one embodiment, therefore, the present invention provides for probe polynucleotides comprising at least 7 consecutive nucleotides of the sequence set forth in SEQ ID NO:13, or the complement thereof.

Non-limiting examples of suitable probe sequences include sequences that comprise SEQ ID NO: 17 or 19 shown in Table 1, as well as probes comprising at least 7 consecutive nucleotides of any one of SEQ ID NOs:14, 15, 17, 19 or 20, or the complement thereof. In one embodiment of the present invention, the polynucleotide probes comprise at least 7 consecutive nucleotides of SEQ ID NO:17 or 19, or the complement thereof.

Various types of probes known in the art are contemplated by the present invention. For example, the probe may be a hybridization probe, the binding of which to a target nucleotide sequence can be detected using a general DNA binding dye such as ethidium bromide, SYBR® Green, SYBR® Gold and the like. Alternatively, the probe can incorporate one or more detectable labels. Detectable labels are molecules or moieties a property or characteristic of which can be detected directly or indirectly and are chosen such that the ability of the probe to hybridize with its target sequence is not affected. Methods of labelling nucleic acid sequences are well-known in the art (see, for example, Ausubel et al., (1997 & updates) Current Protocols in Molecular Biology, Wiley & Sons, New York).

Labels suitable for use with the probes of the present invention include those that can be directly detected, such as radioisotopes, fluorophores, chemiluminophores, enzymes, colloidal particles, fluorescent microparticles, and the like. One skilled in the art will understand that directly detectable labels may require additional components, such as substrates, triggering reagents, light, and the like to enable detection of the label. The present invention also contemplates the use of labels that are detected indirectly. Indirectly detectable labels are typically specific binding members used in conjunction with a “conjugate” that is attached or coupled to a directly detectable label. Coupling chemistries for synthesising such conjugates are well-known in the art and are designed such that the specific binding property of the specific binding member and the detectable property of the label remain intact. As used herein, “specific binding member” and “conjugate” refer to the two members of a binding pair, i.e. two different molecules, where the specific binding member binds specifically to the probe, and the “conjugate” specifically binds to the specific binding member. Binding between the two members of the pair is typically chemical or physical in nature. Examples of such binding pairs include, but are not limited to, antigens and antibodies; avidin/streptavidin and biotin; haptens and antibodies specific for haptens; complementary nucleotide sequences; enzyme cofactors/substrates and enzymes; and the like.

In one embodiment of the present invention, the probe is labelled with a fluorophore. The probe may additionally incorporate a quencher for the fluorophore. Fluorescently labelled probes can be particularly useful for the real-time detection of target nucleotide sequences in a test sample. Examples of probes that are labelled with both a fluorophore and a quencher that are contemplated by the present invention include, but are not limited to, molecular beacon probes and TaqMan® probes. Such probes are well known in the art (see for example, U.S. Pat. Nos. 6,150,097; 5,925,517 and 6,103,476; Marras et al., “Genotyping single nucleotide polymorphisms with molecular beacons.” In Kwok, P. Y. (ed.), “Single nucleotide polymorphisms: methods and protocols,” Vol. 212, pp. 111-128, Humana Press, Totowa, N.J.)

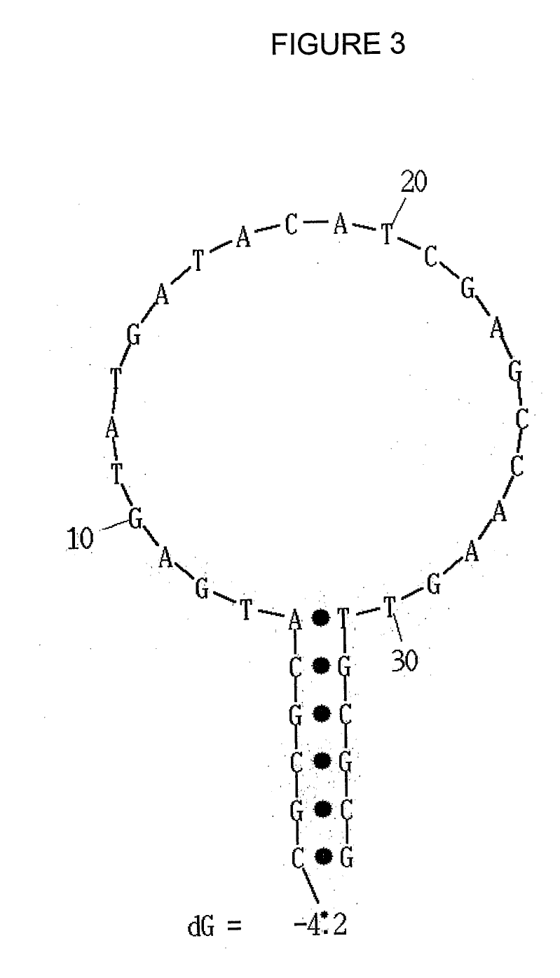

A molecular beacon probe is a hairpin shaped oligonucleotide sequence, which undergoes a conformational change when it hybridizes to a perfectly complementary target sequence. The secondary structure of a typical molecular beacon probe includes a loop sequence, which is capable of hybridizing to a target sequence and a pair of arm (or “stem”) sequences. One arm is attached to a fluorophore, while the other arm is attached to a quencher. The arm sequences are complementary to each other so as to enable the arms to hybridize together to form a molecular duplex and the beacon adopts a hairpin conformation in which the fluorophore and quencher are in close proximity and interact such that emission of fluorescence is prevented. Hybridization between the loop sequence and the target sequence forces the molecular beacon probe to undergo a conformational change in which arm sequences are forced apart and the fluorophore is physically separated from the quencher. As a result, the fluorescence of the fluorophore is restored. The fluorescence generated can be monitored and related to the presence of the target nucleotide sequence. If no target sequence is present in the sample, no fluorescence will be observed. This methodology, as described further below, can also be used to quantify the amount of target nucleotide in a sample. By way of example, FIG. 3 depicts the secondary structure of an exemplary hairpin loop molecular beacon (molecular beacon #1) having a sequence corresponding to SEQ ID NO:16.

Wavelength-shifting molecular beacon probes which incorporate two fluorophores, a “harvester fluorophore and an “emitter” fluorophore (see, Kramer, et al., (2000) Nature Biotechnology, 18:1191-1196) are also contemplated. When a wavelength-shifting molecular beacon binds to its target sequence and the hairpin opens, the energy absorbed by the harvester fluorophore is transferred by fluorescence resonance energy transfer (FRET) to the emitter, which then fluoresces. Wavelength-shifting molecular beacons are particularly suited to multiplex assays.

TaqMan® probes are dual-labelled fluorogenic nucleic acid probes that function on the same principles as molecular beacons. TaqMan® probes are composed of a polynucleotide that is complementary to a target sequence and is labelled at the 5′ terminus with a fluorophore and at the 3′ terminus with a quencher. TaqMan® probes, like molecular beacons, are typically used as real-time probes in amplification reactions. In the free probe, the close proximity of the fluorophore and the quencher ensures that the fluorophore is internally quenched. During the extension phase of the amplification reaction, the probe is cleaved by the 5′ nuclease activity of the polymerase and the fluorophore is released. The released fluorophore can then fluoresce and produce a detectable signal.

Linear probes comprising a fluorophore and a high efficiency dark quencher, such as the Black Hole Quenchers (BHQ™; Biosearch Technologies, Inc., Novato, Calif.) are also contemplated. As is known in the art, the high quenching efficiency and lack of native fluorescence of the BHQ™ dyes allows “random-coil” quenching to occur in linear probes labelled at one terminus with a fluorophore and at the other with a BHQ™ dye thus ensuring that the fluorophore does not fluoresce when the probe is in solution. Upon binding its target sequence, the probe stretches out spatially separating the fluorophore and quencher and allowing the fluorophore to fluoresce. One skilled in the art will appreciate that the BHQ™ dyes can also be used as the quencher moiety in molecular beacon or TaqMan® probes.

As an alternative to including a fluorophore and a quencher in a single molecule, two fluorescently labelled probes that anneal to adjacent regions of the target sequence can be used. One of these probes, a donor probe, is labelled at the 3′ end with a donor fluorophore, such as fluorescein, and the other probe, the acceptor probe, is labelled at the 5′ end with an acceptor fluorophore, such as LC Red 640 or LC Red 705. When the donor fluorophore is stimulated by the excitation source, energy is transferred to the acceptor fluorophore by FRET resulting in the emission of a fluorescent signal.

In addition to providing primers and probes as separate molecules, the present invention also contemplates polynucleotides that are capable of functioning as both primer and probe in an amplification reaction. Such combined primer/probe polynucleotides are known in the art and include, but are not limited to, Scorpion probes, duplex Scorpion probes, Lux™ primers and Amplifluor™ primers.

Scorpion probes consist of, from the 5′ to 3′ end, (i) a fluorophore, (ii) a specific probe sequence that is complementary to a portion of the target sequence and is held in a hairpin configuration by complementary stem loop sequences, (iii) a quencher, (iv) a PCR blocker (such as, hexethylene glycol) and (v) a primer sequence. After extension of the primer sequence in an amplification reaction, the probe folds back on itself so that the specific probe sequence can bind to its complement within the same DNA strand. This opens up the hairpin and the fluorophore can fluoresce. Duplex Scorpion probes are a modification of Scorpion probes in which the fluorophore-coupled probe/primer containing the PCR blocker and the quencher-coupled sequence are provided as separate complementary polynucleotides. When the two polynucleotides are hybridized as a duplex molecule, the fluorophore is quenched. Upon dissociation of the duplex when the primer/probe binds the target sequence, the fluorophore and quencher become spatially separated and the fluorophore fluoresces.

The Amplifluor Universal Detection System also employs fluorophore/quencher combinations and is commercially available from Chemicon International (Temecula, Calif.).

In contrast, Lux™ primers incorporate only a fluorophore and adopt a hairpin structure in solution that allows them to self-quench. Opening of the hairpin upon binding to a target sequence allows the fluorophore to fluoresce.

Suitable fluorophores and/or quenchers for use with the polynucleotides of the present invention are known in the art (see for example, Tyagi et al., Nature Biotechnol., 16:49-53 (1998); Marras et al., Genet. Anal.: Biomolec. Eng., 14:151-156 (1999)). Many fluorophores and quenchers are available commercially, for example from Molecular Probes (Eugene, Oreg.) or Biosearch Technologies, Inc. (Novato, Calif.). Examples of fluorophores that can be used in the present invention include, but are not limited to, fluorescein and fluorescein derivatives, such as 6-carboxyfluoroscein (FAM), 5′-tetrachlorofluorescein phosphoroamidite (TET), tetrachloro-6-carboxyfluoroscein, VIC and JOE, 5-(2′-aminoethyl)aminonaphthalene-1-sulphonic acid (EDANS), coumarin and coumarin derivatives, Lucifer yellow, Texas red, tetramethylrhodamine, 5-carboxyrhodamine, cyanine dyes (such as Cy5) and the like. Pairs of fluorophores suitable for use as FRET pairs include, but are not limited to, fluorescein/rhodamine, fluorescein/Cy5, fluorescein/Cy5.5, fluorescein/LC Red 640, fluorescein/LC Red 750, and phycoerythrin/Cy7. Quenchers include, but are not limited to, 4′-(4-dimethylaminophenylazo)benzoic acid (DABCYL), 4-dimethylaminophenylazophenyl-4′-maleimide (DABMI), tetramethylrhodamine, carboxytetramethylrhodamine (TAMRA), BHQ™ dyes and the like.

Methods of selecting appropriate sequences for and preparing the various primers and probes are known in the art. For example, the polynucleotides can be prepared using conventional solid-phase synthesis using commercially available equipment, such as that available from Applied Biosystems USA Inc. (Foster City, Calif.), DuPont, (Wilmington, Del.), or Milligen (Bedford, Mass.). Methods of coupling fluorophores and quenchers to nucleic acids are also in the art.

In one embodiment of the present invention, the probe polynucleotide is a molecular beacon. In general, in order to form a hairpin structure effectively, molecular beacons are at least 17 nucleotides in length. In accordance with this aspect of the invention, therefore, the molecular beacon probe is typically between about 17 and about 40 nucleotides in length. Within the probe, the loop sequence that corresponds to or is complementary to the target sequence typically is about 7 to about 32 nucleotides in length, while the stem (or “arm”) sequences are each between about 4 and about 9 nucleotides in length. As indicated above, part of the stem sequences of a molecular beacon may also be complementary to the target sequence. In one embodiment of the present invention, the loop sequence of the molecular beacon is between about 10 and about 30 nucleotides in length. In other embodiments, the loop sequence of the molecular beacon is between about 15 and about 30 nucleotides in length.

In accordance with the present invention, the loop region of the molecular beacon probe comprises at least 7 consecutive nucleotides of the sequence as set forth in SEQ ID NO:12, or the complement thereof. In a specific embodiment, the loop region of the molecular beacon probe comprises at least 7 consecutive nucleotides of the sequence as set forth in SEQ ID NO:13, or the complement thereof.

### Amplification and Detection

In accordance with the present invention, S. aureus detection involves subjecting a test sample to an amplification reaction in order to obtain an amplification product, or amplicon, comprising the target sequence.

As used herein, an “amplification reaction” refers to a process that increases the number of copies of a particular nucleic acid sequence by enzymatic means. Amplification procedures are well-known in the art and include, but are not limited to, polymerase chain reaction (PCR), TMA, rolling circle amplification, nucleic acid sequence based amplification (NASBA), strand displacement amplification (SDA) and Q-beta replicase amplification. One skilled in the art will understand that for use in certain amplification techniques the primers described above may need to be modified, for example, SDA primers comprise additional nucleotides near the 5′ end that constitute a recognition site for a restriction endonuclease. Similarly, NASBA primers comprise additional nucleotides near the 5′ end that are not complementary to the target sequence but which constitute an RNA polymerase promoter. Polynucleotides thus modified are considered to be within the scope of the present invention.

In one embodiment of the present invention, the target sequence is amplified by PCR. PCR is a method known in the art for amplifying a nucleotide sequence using a heat stable polymerase and a pair of primers, one primer (the forward primer) complementary to the (+)-strand at one end of the sequence to be amplified and the other primer (the reverse primer) complementary to the (−)-strand at the other end of the sequence to be amplified. Newly synthesized DNA strands can subsequently serve as templates for the same primer sequences and successive rounds of strand denaturation, primer annealing, and strand elongation, produce rapid and highly specific amplification of the target sequence. PCR can thus be used to detect the existence of a defined sequence in a DNA sample. The term “PCR” as used herein refers to the various forms of PCR known in the art including, but not limited to, quantitative PCR, reverse-transcriptase PCR, real-time PCR, hot start PCR, long PCR, LAPCR, multiplex PCR, touchdown PCR, and the like. “Real-time PCR” refers to a PCR reaction in which the amplification of a target sequence is monitored in real time by, for example, the detection of fluorescence emitted by the binding of a labelled probe to the amplified target sequence.

The present invention thus provides for amplification of a portion of a S. aureus femB gene of less than about 500 nucleotides in length and comprising at least 55 consecutive nucleotides of the sequence set forth in SEQ ID NO:12 using a pair of polynucleotide primers, each member of the primer pair comprising at least 7 consecutive nucleotides of the sequence as set forth in SEQ ID NO:1, or the complement thereof.

The product of the amplification reaction can be detected by a number of means known to individuals skilled in the art. Examples of such detection means include, for example, gel electrophoresis and/or the use of polynucleotide probes. In one embodiment of the invention, the amplification products are detected through the use of polynucleotide probes. Such polynucleotide probes are described in detail above.

A further embodiment of the invention, therefore, provides for amplification and detection of a portion of a S. aureus femB gene of less than about 500 nucleotides in length and comprising at least 55 consecutive nucleotides of the sequence set forth in SEQ ID NO:12 using a combination of polynucleotides, the combination comprising one or more polynucleotide primers comprising at least 7 consecutive nucleotides of the sequence as set forth in SEQ ID NO:1, or the complement thereof, and a polynucleotide probe comprising at least 7 consecutive nucleotides of the sequence as set forth in SEQ ID NO:12, or the complement thereof.

It will be readily appreciated that a procedure that allows both amplification and detection of target S. aureus nucleic acid sequences to take place concurrently in a single unopened reaction vessel would be advantageous. Such a procedure would avoid the risk of “carry-over” contamination in the post-amplification processing steps, and would also facilitate high-throughput screening or assays and the adaptation of the procedure to automation. Furthermore, this type of procedure allows “real time” monitoring of the amplification reaction, as discussed above, as well as more conventional “end-point” monitoring. In one embodiment, the detection is accomplished in real time in order to facilitate rapid detection. In a specific embodiment, detection is accomplished in real time through the use of a molecular beacon probe.

The present invention thus provides for methods to specifically amplify and detect S. aureus nucleic acid sequences in a test sample in a single tube format using the polynucleotide primers, and optionally one or more probes, described herein. Such methods may employ dyes, such as SYBR® Green or SYBR® Gold that bind to the amplified target sequence, or an antibody that specifically detects the amplified target sequence. The dye or antibody is included in the reaction vessel and detects the amplified sequences as it is formed. Alternatively, a labelled polynucleotide probe (such as a molecular beacon or TaqMan® probe) distinct from the primer sequences, which is complementary to a region of the amplified sequence, may be included in the reaction, or one of the primers may act as a combined primer/probe, such as a Scorpion probe. Such options are discussed in detail above.

Thus, a general method of detecting S. aureus in a sample is provided that comprises contacting a test sample suspected of containing, or known to contain, an S. aureus target nucleotide sequence with a combination of polynucleotides comprising at least one polynucleotide primer and at least one polynucleotide probe or primer/probe, as described above, under conditions that permit amplification of said target sequence, and detecting any amplified target sequence as an indication of the presence of S. aureus in the sample. A “test sample” as used herein is a biological sample suspected of containing, or known to contain, a S. aureus target nucleotide sequence.

In one embodiment of the present invention, a method using the polynucleotide primers and probes or primer/probes is provided to specifically amplify and detect a S. aureus target nucleotide sequence in a test sample, the method generally comprising the steps of:
(a) forming a reaction mixture comprising a test sample, amplification reagents, one or more polynucleotide probes capable of specifically hybridising to a portion of a S. aureus target nucleotide sequence and one or more polynucleotide primers corresponding to or complementary to a portion of a S. aureus femB gene comprising said target nucleotide sequence;
(b) subjecting the mixture to amplification conditions to generate at least one copy of the target nucleotide sequence, or a nucleic acid sequence complementary to the target nucleotide sequence;
(c) hybridizing the probe to the target nucleotide sequence or the nucleic acid sequence complementary to the target sequence, so as to form a probe:target hybrid; and
(d) detecting the probe:target hybrid as an indication of the presence of the S. aureus target nucleotide sequence in the test sample.

In one embodiment of the present invention, the method employs one or more labelled probes in step (a).

The term “amplification reagents” includes conventional reagents employed in amplification reactions and includes, but is not limited to, one or more enzymes having nucleic acid polymerase activity, enzyme cofactors (such as magnesium or nicotinamide adenine dinucleotide (NAD)), salts, buffers, nucleotides such as deoxynucleotide triphosphates (dNTPs; for example, deoxyadenosine triphosphate, deoxyguanosine triphosphate, deoxycytidine triphosphate and deoxythymidine triphosphate) and other reagents that modulate the activity of the polymerase enzyme or the specificity of the primers.

It will be readily understood by one skilled in the art that step (b) of the above method can be repeated several times prior to step (c) by thermal cycling the reaction mixture by techniques known in the art and that steps (b), (c) and (d) may take place concurrently such that the detection of the amplified sequence takes place in real time. In addition, variations of the above method can be made depending on the intended application of the method, for example, the polynucleotide probe may be a combined primer/probe, or it may be a separate polynucleotide probe, in which case two different polynucleotide primers are used. Additional steps may be incorporated before, between or after those listed above as necessary, for example, the test sample may undergo enrichment, extraction and/or purification steps to isolate nucleic acids therefrom prior to the amplification reaction, and/or the amplified product may be submitted to purification/isolation steps or further amplification prior to detection, and/or the results from the detection step (d) may be analysed in order to quantify the amount of target present in the sample or to compare the results with those from other samples. These and other variations will be apparent to one skilled in the art and are considered to be within the scope of the present invention.

In one embodiment of the present invention, the method is a real-time PCR assay utilising two polynucleotide primers and a molecular beacon probe.

### Diagnostic Assays to Detect S. aureus

The present invention provides for diagnostic assays using the polynucleotide primers and/or probes that can be used for highly specific detection of S. aureus in a test sample. The diagnostic assays comprise amplification and detection of S. aureus nucleic acids as described above. The diagnostic assays can be qualitative or quantitative and can involve real-time monitoring of the amplification reaction or conventional end-point monitoring.

In one embodiment, the invention provides for diagnostic assays that do not require post-amplification manipulations and minimise the amount of time required to conduct the assay. For example, in a specific embodiment, there is provided a diagnostic assay, utilising the primers and probes described herein, that can be completed using real time PCR technology in about 54 hours and generally in 24 hours or less.

Such diagnostic assays are particularly useful in the detection of S. aureus contamination of various foodstuffs. Thus, in one embodiment, the present invention provides a rapid and sensitive diagnostic assay for the detection of S. aureus contamination of a food sample. S. aureus contamination is common on foods that require a considerable amount of handling during preparation and that are kept at a slightly elevated temperature after preparation. Foods that can be analysed using the diagnostic assays include, but are not limited to, dairy products such as milk, including raw milk, cheese, yoghurt, ice cream and cream; raw, cooked and cured meats and meat products, such as beef, pork, lamb, mutton, poultry (including turkey, chicken), game (including rabbit, grouse, pheasant, duck), minced and ground meat (including ground beef, ground turkey, ground chicken, ground pork); eggs; fruits and vegetables; nuts and nut products, such as nut butters; seafood products including fish and shellfish; fruit or vegetable juices; bakery products, including bread, cakes, pastries, pies and cream-filled baked goods, and prepared foods, such as egg dishes, pastas and salads, including egg, tuna, chicken, potato and pasta salads. The diagnostic assays may also be used to detect S. aureus contamination of drinking water.

While the primary focus of S. aureus detection is food products, the present invention also contemplates the use of the primers and probes in diagnostic assays for the detection of S. aureus contamination of other biological samples, such as patient specimens in a clinical setting, for example, faeces, blood, saliva, throat swabs, urine, mucous, and the like, as well as S. aureus contamination of surfaces and instruments, such as surgical or dental instruments. The diagnostic assays are also useful in the assessment of microbiologically pure cultures, and in environmental and pharmaceutical quality control processes.

The test sample can be used in the assay either directly (i.e. as obtained from the source) or following one or more pre-treatment steps to modify the character of the sample. Thus, the test sample can be pre-treated prior to use, for example, by disrupting cells or tissue, extracting the microbial content from the sample (such as a swab or wipe test sample), enhancing/enriching the microbial content of the sample by culturing in a suitable medium, preparing liquids from solid materials, diluting viscous fluids, filtering liquids, distilling liquids, concentrating liquids, inactivating interfering components, adding reagents, isolating nucleic acids, purifying nucleic acids, and the like. In one embodiment of the present invention, the test sample is subjected to one or more steps to isolate, or partially isolate, nucleic acids therefrom. In another embodiment of the invention, the test sample is subjected to an enrichment procedure to enhance the microbial content of the sample prior to use in the assay.

As indicated above, the polynucleotide primers and probes of the invention can be used in assays to quantitate the amount of a S. aureus target nucleotide sequence in a test sample. Thus, the present invention provides for a method to specifically amplify, detect and quantitate a target nucleotide sequence in a test sample, the methods generally comprising the steps of:
(a) forming a reaction mixture comprising a test sample, amplification reagents, one or more polynucleotide probes capable of specifically hybridising to a portion of a S. aureus target nucleotide sequence and one or more polynucleotide primers corresponding to or complementary to a portion of a S. aureus femB gene comprising said target nucleotide sequence;
(b) subjecting the mixture to amplification conditions to generate at least one copy of the target nucleotide sequence, or a nucleic acid sequence complementary to the target nucleotide sequence;
(c) hybridizing the probe to the target nucleotide sequence or the nucleic acid sequence complementary to the target sequence, so as to form a probe:target hybrid;
(d) detecting the probe:target hybrid; and
(e) analysing the amount of probe:hybrid present as an indication of the amount of target nucleotide sequence present in the test sample.

The steps of this method may also be varied as described above for the amplification/detection method.

In one embodiment, the method employs one or more labelled polynucleotide probes in step (a) and steps (d) and (e) are as follows:
(d) detecting the probe:target hybrid by detecting the signal produced by the hybridized labelled probe; and
(e) analysing the amount of signal produced as an indication of the amount of target nucleotide sequence present in the test sample.

Step (e) can be conducted, for example, by comparing the amount of signal produced to a standard or utilising one of a number of statistical methods known in the art that do not require a standard.

Various types of standards for quantitative assays are known in the art. For example, the standard can consist of a standard curve compiled by amplification and detection of known quantities of the S. aureus target nucleotide sequence under the assay conditions. Alternatively, relative quantitation can be performed without the need for a standard curve (see, for example, Pfaffl, M W. (2001) Nucleic Acids Research 29(9):2002-2007). In this method, a reference gene is selected against which the expression of the target gene can be compared and an additional pair of primers and an appropriate probe are included in the reaction in order to amplify and detect a portion of the selected reference gene. The reference gene is usually a gene that is expressed constitutively, for example, a house-keeping gene.

Another similar method of quantification is based on the inclusion of an internal standard in the reaction. Such internal standards generally comprise a control target nucleotide sequence and a control polynucleotide probe. The internal standard can further include an additional pair of primers that specifically amplify the control target nucleotide sequence and are unrelated to the polynucleotides of the present invention. Alternatively, the control target sequence can contain primer target sequences that allow specific binding of the assay primers but a different probe target sequence. This allows both the S. aureus target sequence and the control sequence to be amplified with the same primers, but the amplicons are detected with separate probe polynucleotides. Typically, when a reference gene or an internal standard is employed, the reference/control probe incorporates a detectable label that is distinct from the label incorporated into the S. aureus target sequence specific probe. The signals generated by these two labels when they bind their respective target sequences can thus be distinguished.

In the context of the present invention, a control target nucleotide sequence is a nucleic acid sequence that (i) can be amplified either by the S. aureus target sequence specific primers or by control primers, (ii) specifically hybridizes to the control probe under the assay conditions and (iii) does not exhibit significant hybridization to the S. aureus target sequence specific probe under the same conditions. One skilled in the art will recognise that the actual nucleic acid sequences of the control target nucleotide and the control probe are not important provided that they both meet the criteria outlined above.

The diagnostic assays can be readily adapted for high-throughput. High-throughput assays provide the advantage of processing many samples simultaneously and significantly decrease the time required to screen a large number of samples. The present invention, therefore, contemplates the use of the polynucleotides of the present invention in high-throughput screening or assays to detect and/or quantitate S. aureus target nucleotide sequences in a plurality of test samples.

For high-throughput assays, reaction components are usually housed in a multi-container carrier or platform, such as a multi-well microtitre plate, which allows a plurality of assays each containing a different test sample to be monitored simultaneously. Control samples can also be included in the plates to provide internal controls for each plate. Many automated systems are now available commercially for high-throughput assays, as are automation capabilities for procedures such as sample and reagent pipetting, liquid dispensing, timed incubations, formatting samples into microarrays, microplate thermocycling and microplate readings in an appropriate detector, resulting in much faster throughput times.

### Kits and Packages for the Detection of S. aureus

The present invention further provides for kits for detecting S. aureus in a variety of samples. In general, the kits comprise a pair of primers and a probe capable of amplifying and detecting an S. aureus target sequence as described above. One of the primers and the probe may be provided in the form of a single polynucleotide, such as a Scorpion probe, as described above. The probe provided in the kit can be unlabelled, or can incorporate a detectable label, such as a fluorophore or a fluorophore and a quencher, or the kit may include reagents for labelling the probe. The primers/probes can be provided in separate containers or in an array format, for example, pre-dispensed into microtitre plates.

The kits can optionally include amplification reagents, such as buffers, salts, enzymes, enzyme co-factors, nucleotides and the like. Other components, such as buffers and solutions for the enrichment, isolation and/or lysis of bacteria in a test sample, extraction of nucleic acids, purification of nucleic acids and the like may also be included in the kit. One or more of the components of the kit may be lyophilised and the kit may further comprise reagents suitable for the reconstitution of the lyophilised components. The lyophilised components may further comprise additives that facilitate their reconstitution.

The various components of the kit are provided in suitable containers. As indicated above, one or more of the containers may be a microtitre plate. Where appropriate, the kit may also optionally contain reaction vessels, mixing vessels and other components that facilitate the preparation of reagents or nucleic acids from the test sample.

The kit may additionally include one or more controls. For example, control polynucleotides (primers, probes, target sequences or a combination thereof) may be provided that allow for quality control of the amplification reaction and/or sample preparation, or that allow for the quantitation of S. aureus target nucleotide sequences.

The kit can additionally contain instructions for use, which may be provided in paper form or in computer-readable form, such as a disc, CD, DVD or the like.

The present invention further contemplates that the kits described above may be provided as part of a package that includes computer software to analyse data generated from the use of the kit.

The invention will now be described with reference to specific examples. It will be understood that the following examples are intended to describe preferred embodiments of the invention and are not intended to limit the invention in any way.

### EXAMPLES

### Example 1

### Determination of Unique, Conserved DNA Regions in S. aureus Group

The femB gene coding regions from 10 different S. aureus isolates were sequenced and aligned using the multiple alignment program Clustal W™. The resulting alignment was used to identify short DNA regions that were conserved within the S. aureus group, yet which are excluded from other bacteria. FIG. 1 depicts a sample of such an alignment in which a portion of the femB gene of 10 different S. aureus isolates (corresponding to SEQ ID NOs:2-11) have been aligned.

An 85 nucleotide conserved sequence was identified as described above (SEQ ID NO:12).

5′-CR*CATGGTTACGAGCATCATGGCTTTACAACTGAGTATGATACATC
GAGCCAAGTACGATGGATGGGCGTATTAAACCTTGAAGG-3′

This unique and conserved element of S. aureus femB-gene sequences (consensus sequence) was used to design highly specific primers for the PCR amplification of a conserved region of the femB gene.

### Example 2

### Generation of PCR Primers for Amplication of the femB Consensus Sequence

Within the conserved 85 nucleotide sequence identified as described in Example 1, two regions that could serve as primer target sequences were identified. These primer target sequences were used to design a pair of primers to allow efficient PCR amplification. The primer sequences are shown below:

| | |
|---|---|
| Forward primer: | |
| 5′-CACATGGTTACGAGCATC-3′ | [SEQ ID NO:14] |
| Reverse primer: | |
| 5′-CCTTCAAGGTTTAATACGC-3′ | [SEQ ID NO:15] |

In the alignment presented in FIG. 1, the positions of the forward and reverse primers are represented by shaded boxes. The forward primer starts at position 464 and ends at position 481 of the alignment. The reverse primer represents the reverse complement of the region starting at position 530 and ending at position 548.

As shown in FIG. 1, the second nucleotide in the forward primer sequence [SEQ ID NO:14] can be either an A or a G. Thus, a degenerate primer could also be used as a forward primer in place of SEQ ID NO:14. Such a degenerate primer would have the sequence:

| | |
|---|---|
| 5′-CR*CATGGTTACGAGCATC-3′ | [SEQ ID NO:20] |

wherein R represents either A or G.

### Example 3

### Generation of Molecular Beacon Probes Specific for S. aureus

In order to design molecular beacon probes specific for S. aureus, a region within the consensus sequence described above was identified which not only was highly conserved in all S. aureus isolates but was also exclusive to S. aureus isolates. This sequence consisted of a 24 nucleotide region that would be suitable for use as a molecular beacon target sequence. The sequence is provided below:

| | |
|---|---|
| 5′-TGAGTATGATACATCGAGCCAAGT-3′ | [SEQ ID NO:13] |

The complement of this sequence [SEQ ID NO:19, see Table 1] is also suitable for use as a molecular beacon target sequence.

A molecular beacon probe having the sequence shown below was synthesized by Integrated DNA Technologies Inc.

### Molecular Beacon Probe #1:

[SEQ ID NO:16]

5′-cgcgcaTGAGTATGATACATCGAGCCAAGTtgcgcg-3′

The complement of this sequence (SEQ ID NO:18, shown below) can also be used as a molecular beacon probe for detecting S. aureus.

[SEQ ID NO:18]

5′-cgcgcaACTTGGCTCGATGTATCATACTCAtgcgcg-3′

The starting material for the synthesis of the molecular beacons was an oligonucleotide that contains a sulfhydryl group at its 5′ end and a primary amino group at its 3′ end. DABCYL was coupled to the primary amino group utilizing an amine-reactive derivative of DABCYL. The oligonucleotides that were coupled to DABCYL were then purified. The protective trityl moiety was then removed from the 5′-sulfhydryl group and a fluorophore was introduced in its place using an iodoacetamide derivative.

An individual skilled in the art would recognize that a variety of methodologies could be used for synthesis of the molecular beacons. For example, a controlled-pore glass column that introduces a DABCYL moiety at the 3′ end of an oligonucleotide has recently become available, which enables the synthesis of a molecular beacon completely on a DNA synthesizer.

Table 2 provides a general overview of the characteristics of molecular beacon probe #1. The beacon sequence shown in Table 2 indicates the stem region in lower case and the loop region in upper case.

TABLE 2
Description of molecular beacon probe #1.

Beacon sequence (5′→3′):
cgcgcaTGAGTATGATACATCGAGCCAAGTtgcgcg
Fluorophore (5′): FAM
Quencher (3′): DABCYL
Specificity: Staphylococcus aureus

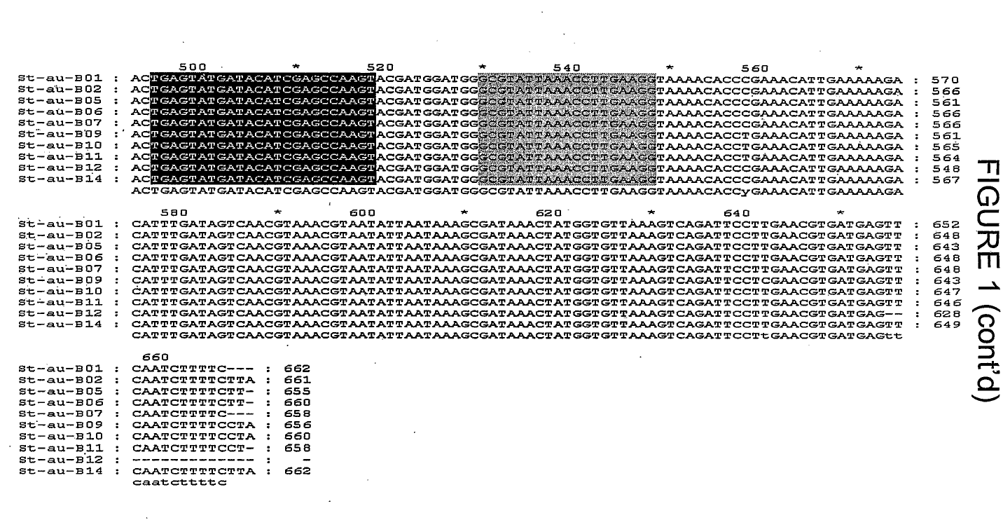

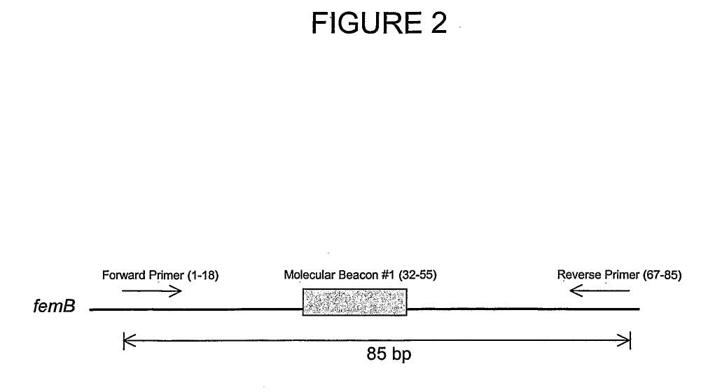

Table 3 provides an overview of the thermodynamics of the folding of molecular beacon probe #1. Calculations were made using MFOLD™ software, or the Oligo Analyzer software package available on Integrated DNA Technologies Inc. web site. FIG. 2 shows the arrangement of PCR primers (SEQ ID NOs:14 and 15) and the molecular beacon probe #1 in the femB consensus sequence. Numbers in parentheses indicate the positions of the first and last nucleotides of each feature on the PCR product generated with the forward and reverse primers.

TABLE 3
Thermodynamics of molecular beacon probe #1.

| | | |
|---|---|---|
| Tm loop (thermodynamics algorithm) | 57.2° | C. |
| Tm stem (mFOLD calculation) | 64.4° | C. |
| ΔG₃₇ (mFOLD calculation) | −4.17 | kCal/mol |
| ΔH (mFOLD calculation) | −51.7 | kCal/mol |

### Example 4

### Isolation of DNA from Samples

The following protocol was utilized in order to isolate DNA sequences from samples.

Material needed for DNA extraction:
Tungsten carbide beads: Qiagen
Reagent DX: Qiagen
DNeasy Plant Mini Kit: Qiagen
Tissue Disruption equipment: Mixer Mill™ 300 (Qiagen)

The following method was followed:
1) Add to a 2 ml screw top tube: 1 tungsten carbide bead and 0.1 g glass beads 212 to 300 μm in width+sample to be analysed+500 μL of AP1 buffer+1 μL of Reagent DX+1 μL of RNase A (100 mg/mL). Extraction control done without adding sample to be analysed.
2) heat in Dry-Bath at 80° C. for 10 min.
3) mix in a Mixer Mill 300 (MM300) at frequency of 30 Hz [1/s], 2 min.
4) rotate tubes and let stand for 10 min at room temperature.
5) mix in a Mixer Mill 300, frequency 30 Hz, 2 min.
6) place tubes in boiling water for 5 min.
7) centrifuge with a quick spin.
8) add 150 μL of AP2 buffer.
9) mix at frequency of 30 Hz for 30 sec. Rotate tubes and repeat.
10) centrifuge at 13,000 rpm for 1 min.
11) place tubes at −20° C. for 10 min.
12) centrifuge at 13,000 rpm for 1 min.
13) transfer supernatant in to a 2 mL screw top tube containing 850 μL of AP3/E buffer.
14) mix by inverting, centrifuge with a quick spin.
15) add 700 μL of mixture. From step 13 to a DNeasy binding column and centrifuge at 800 rpm for 1 minute. Discard eluted buffer. Repeat process with leftover mixture from step 13.
16) add 500 μL of wash buffer (AW buffer) to binding columns and centrifuge for 1 minute at 800 rpm. Discard eluted buffer.
17) add 500 μL of wash buffer (AW buffer) to binding columns and centrifuge for 1 minute at 800 rpm. Discard eluted buffer.
18) centrifuge column again at 8000 rpm for 1 min.
19) place column in a sterile 2 mL tube and add 50 μL of AE elution buffer preheated at 80° C.
20) incubate for 1 min. Centrifuge at max speed for 2 min. Elute twice with 50 μL; final volume should be 100 μL.
21) keep elution for PCR amplification.
   Time of manipulation: 3 hours. Proceed to prepare PCR reaction for real-time detection.

### Example 5

### Amplification of a Target Sequence and Hybridization of Molecular Beacon Probe #1 in Real Time

PCR amplification was undertaken using the conditions described in Tables 4 and 5 below. The intensity of fluorescence emitted by the fluorophore component of the molecular beacon was detected at the annealing stage of each amplification cycle. In Table 4, note that the PCR buffer contains 1.5 mM magnesium chloride (final concentration). Inclusion of additional magnesium chloride brings the final concentration to 4 mM in the reaction mixture.

TABLE 4
PCR Mix used for Validations

| | Final concentration in |
|---|---|
| Reagent | reconstituted reaction |
| Qiagen PCR buffer, 10X | 1.5 X |
| Forward primer [SEQ ID NO: 14], 25 μM | 0.5 μM |
| Reverse primer [SEQ ID NO: 15], 25 μM | 0.5 μM |
| dNTPs, 10 mM | 0.2 mM |
| MgCl₂, 25 mM | 1.75 mM |
| Molecular beacon #1, 10 μM | 0.3 μM |
| HotStarTaq, 5 U/μL | 1 U/25 μL reaction |

Table 5 presents an overview of the cycles used for each step of the PCR amplification.

TABLE 5
PCR Program used Throughout Diagnostic Test Validations

| Step | Temperature | Duration | Repeats |
|---|---|---|---|
| Initial polymerase activation | 95° C. | 15 min | 1 |
| Denaturation | 94° C. | 15 sec | 40 |
| Annealing | 55° C. | 15 sec | |
| Elongation | 72° C. | 15 sec | |

Fluorescence was detected in real-time using a fluorescence monitoring real-time PCR instrument, for example, a BioRad iCycler iQ™ or MJ Research Opticon™. Other instruments with similar fluorescent reading abilities can also be used.

### Example 6

### Quantification of Target Sequence in a Sample

In order to quantify the amount of target sequence in a sample, DNA was isolated and amplified as described in the preceding Examples (4 and 5). DNA was quantified using a standard curve constructed from serial dilutions of a target DNA solution of known concentration.

### Example 7

### Positive Validation for the Specificity of Molecular Beacon Probe #1 for Detection of S. aureus

Genomic DNA from the 25 strains of S. aureus was isolated and amplified as described in the preceding Examples (4 and 5). When no probe was included in the amplification reaction, any amplicons produced were detected using SYBR® Green. All 25 strains of S. aureus were detected.

Also included in additional rounds of tests was molecular beacon probe #1. The molecular beacon probe #1 was capable of detecting all S. aureus isolates tested.

If required, an upper Ct limit can be employed in the assay. Suitable exemplary upper limits appropriate for an assay using the primers SEQ ID NOs:14 and 15 and molecular beacon probe #1 with the PCR protocol outlined above, are between about 35 and 38 Ct, for example 37 Ct.

### Example 8

### Negative Validation of the Primers and Molecular Beacon Probe #1

In order to test the ability of molecular beacon probe #1 to preferentially detect only S. aureus, a number of bacteria other than S. aureus, including other Staphylococcal species, were tested, as generally described below.

Samples of genomic DNA from the bacteria presented in Table 6 below were isolated and amplified as described in the preceding Examples (4 and 5). When no probe was included in the amplification reaction, any amplicons produced were detected using SYBR® Green. No amplification products were observed.

Also included in additional rounds of tests was molecular beacon probe #1. No hybridization of this molecular beacon was observed.

If required, an upper Ct limit can be employed in the assay. Suitable exemplary upper limits appropriate for an assay using the primers SEQ ID NOs:14 and 15 and molecular beacon probe #1 with the PCR protocol outlined above, are between about 35 and 38 Ct, for example 37 Ct.

In Table 6, the figures in parentheses indicate the number of strains of each species that were tested (if more than one). None of the tested strains provided a positive result.

The above results indicate that the forward and reverse primers (SEQ ID NOs:14 and 15,respectively) and molecular beacon probe #1 (SEQ ID NO:16) are highly specific for S. aureus.

TABLE 6
Negative Validation of the Primers and Molecular Beacon Probe #1

| Acinetobacter | Citrobacter koseri | Lactococcus | Salmonella typhi |
|---|---|---|---|
| calcoaceticus (2) | (2) | raffinolactis | (2) |
| Acinetobacter | Citrobacter | Legionella | Salmonella |
| iwoffi | werkmanii | pneumophila | typhimurium (2) |
| Acinetobacter junii | Clostridium | Listeria grayi | Salmonella enterica |
| | botulinum (2) | | subsp. enterica |
| | | | serovar Typhisuis (2) |
| Aeromonas | Clostridium | Listeria innocua (2) | Serratia |
| hydrophila (2) | butyricum | | liquefaciens (2) |
| Aeromonas | Clostridium | Listeria ivanovii (2) | Serratia |
| salmonicida (2) | difficile | | marcescens (2) |
| Alcaligenes faecalis | Clostridium | Listeria | Serratia odorifera |
| | perfringens (2) | monocytogenes (2) | |
| Bacillus | Clostridium | Listeria seeligeri | Shigella boydii |
| amyloliquefaciens | sporogenes | | |
| (2) | | | |
| Bacillus cereus (2) | Clostridium tetani | Listeria welshimeri | Shigella |
| | | (2) | dysenteriae (2) |
| Bacillus circulans | Clostridium | Micrococcus luteus | Shigella flexneri (2) |
| (2) | tyrobutyricum | (2) | |
| Bacillus coagulans | Corynebacterium | Mycobacterium | Shigella sonnei (2) |
| (2) | xerosis | smegmatis | |
| Bacillus firmus | Edwardsiella tarda | Neisseria | Staphylococcus |
| | | gonorrhoeae | auricularis |
| Bacillus lentus | Enterobacter | Neisseria lactamica | Staphylococcus |
| | aerogenes (2) | | caprae (4) |
| Bacillus | Enterobacter | Neisseria | Staphylococcus |
| licheniformis (2) | amnigenus | meningitidis (2) | chromogenes |
| Bacillus | Enterobacter | Neisseria sica | Staphylococcus |
| megaterium (2) | cloacae (2) | | epidermidis (5) |
| Bacillus mycoides | Enterobacter | Nocardia | Staphylococcus |
| | intermedius (2) | asteroides | hyicus (4) |
| Bacillus pumilus | Enterobacter | Pediococcus | Staphylococcus |
| (2) | taylorae | acidilactici (2) | intermedius |
| Bacillus sphaericus | Enterococcus | Pediococcus | Staphylococcus |
| | faecalis (2) | pentosaceus | lentis |
| Bacillus | Enterococcus | Proteus mirabilis | Staphylococcus |
| stearothermophilus | faecium | (2) | ludgdunensis |
| Bacillus subtilis (2) | Enterococcus hirae | Proteus penneri (2) | Staphylococcus |
| | (2) | | spp. |
| Bacillus | Erwinia herbicola | Proteus vulgaris (2) | Staphylococcus |
| thuringiensis (2) | | | schieiferi |
| Bacteroides fragilis | Escherichia blattae | Pseudomonas | Staphylococcus |
| | (2) | aeruginosa (2) | xylosus |
| Bifidobacterium | Escherichia coli (4) | Pseudomonas | Stenotrophomonas |
| adolescentis | | alcaligenes | hyicus |
| Bifidobacterium | Escherichia | Pseudomonas | Stenotrophomonas |
| animalis | fergusonii | mendocina | maltophilia |
| Bifidobacterium | Escherichia | Pseudomonas | Streptococcus |
| bifidum | hermanii (2) | pseudoalcaligenes | agalactiae (2) |
| Bifidobacterium | Escherichia | Pseudomonas | Streptococcus bovis |
| longum | vulneris (2) | putida (2) | |
| Bifidobacterium | Haemophilus | Pseudomonas | Streptococcus |
| pseudolongum | equigenitalis | stutzeri | hyicus |
| Bifidobacterium | Haemophilus | Salmonella | Streptococcus |
| spp. (2) | influenzae (2) | enterica, subsp. | intermedius (4) |
| | | enterica serovar | |
| | | Agona | |
| Bifidobacterium | Haemophilus | Salmonella | Streptococcus |
| suis | paragallinarum | choleraesuis subsp. | pneumoniae (2) |
| | | Arizonae (2) | |
| Bifidobacterium | Hafnia alvei (2) | Salmonella bongori | Streptococcus |
| thermophilus | | | pyogenes (2) |
| Bordetella | Helicobacter pylori | Salmonella | Streptococcus |
| bronchiseptica | | enterica, subsp. | saprophyticus |
| | | enterica serovar | |
| | | Brandenburg | |
| Bordetella pertussis | Klebsiella | Salmonella | Streptococcus |
| | ornithinolytica | choleraesuis (2) | schleiferi |
| Borrelia | Klebsiella oxytoca | Salmonella | Streptococcus suis |
| burgdorferi | (2) | enterica, subsp. | |
| | | diarizonae | |
| Branhamella | Klebsiella | Salmonella enterica | Vibrio alginolyticus |
| catarrhalis | planticola (2) | subsp. enterica | |
| | | serovar Dublin (2) | |
| Brevibacillus | Klebsiella | Salmonella | Vibrio cholerae (2) |
| laterosporus | pneumoniae (2) | enteritidis (2) | |
| Campylobacter coli | Klebsiella terrigena | Salmonella | Vibrio eltor |
| | | enterica, subsp. | |
| | | enterica serovar | |
| | | Heidelberg (2) | |
| Campylobacter | Kocuria kristinae | Salmonella | Vibrio fluvialis |
| jejuni (2) | | enterica, subsp. | |
| | | houtenae | |
| Campylobacter lari | Kurthia zopfii (2) | Salmonella enterica | Vibrio hollisae |
| (2) | | subsp. indica | |
| Campylobacter | Lactobacillus | Salmonella enterica | Vibrio vulnificus |
| rectus | acidophilus | subsp. enterica | |
| | | serovar Infantis (2) | |
| Cellilomonea spp. | Lactobacillus casei | Salmonella enterica | Xanthomonas |
| | (2) | subsp. enterica | campestris |
| | | serovar Montevideo | |
| | | (2) | |
| Chromobacterium | Lactobacillus | Salmonella enterica | Yersinia |
| violaceum | delbreuckii (2) | subsp. enterica | enterocolitica (2) |
| | | serovar Newport | |
| | | (2) | |
| Chryseobacterium | Lactobacillus | Salmonella | Yersinia |
| spp. | helveticus | paratyphi (4) | frederiksenii |
| Chryseomonas | Lactobacillus | Salmonella enterica | Yersinia kritensenii |
| luteola | pentosus | subsp. enterica | |
| | | serovar Saintpaul | |
| | | (2) | |
| Citrobacter | Lactobacillus | Salmonella enterica | |
| amalonaticus (2) | plantarum (2) | subsp. enterica | |
| | | serovar | |
| | | Senftenberg | |
| Citrobacter | Lactobacillus | Salmonella enterica | |
| diversus | rhamnosus (2) | subsp. enterica | |
| | | serovar Stanley | |
| Citrobacter | Lactococcus lactis | Salmonella enterica | |
| freundii (2) | (2) | subsp. enterica | |
| | | serovar Thompson | |
| | | (2) | |

### Example 9

### Enrichment of Test Sample Prior to Use n Assay

Samples to be tested can be enriched prior to use in the assay using standard enrichment procedures. The following is a representative protocol for food samples:
1. Place 10 g or 10 ml of the sample in a stomacher filter bag with 90 ml of Peptone Water 0.1%.
2. Homogenize the contents of the bag for 10 sec using a Stomacher instrument (BagMixer).
3. Transfer the required inoculation volume of the sample from the Stomacher bag into a Baird-Parker Agar tube with Egg Yolk Tellurite Emulsion (E-Y-T Emulsion; Oxoid). The required inoculation volume can be determined using the guidelines provided in Table 7 (based on the regulatory guidelines for food in Canada).
4. Spread the liquid over the surface of the agar by gently rotating the tube.
5. Incubate at 35° C. for 18 hours in a slanted position while keeping the agar surface facing upward.
6. After incubation, add 2 ml of sterile Peptone Water 0.1% to each tube.
7. Vortex to resuspend cells.
8. Transfer 1 ml of the cell suspension into a sterile tube and proceed with DNA extraction (for example, following the protocol in Example 4).

TABLE 7
Regulatory Limits for Food in Canada

| Regulatory Limit | | Secondary | |
|---|---|---|---|
| for Food in | Primary Dilution | Dilution of Food | Volume to be |
| Canada (cfu/g) | of Food Sample | Sample | Inoculated (μL) |
| 10 | 1:10 | N/A | 350 |
| 50 | 1:10 | N/A | 350 |
| 100 | 1:10 | N/A | 350 |
| 250 | 1:10 | N/A | 150 |
| 500 | 1:10 | N/A | 75 |
| 1000 | 1:10 | 1:10 | 350 |

Although the invention has been described with reference to certain specific embodiments, various modifications thereof will be apparent to those skilled in the art without departing from the spirit and scope of the invention as outlined in the claims appended hereto.

The disclosure of all patents, publications, including published patent applications, and database entries referenced in this specification are specifically incorporated by reference in their entirety to the same extent as if each such individual patent, publication, and database entry were specifically and individually indicated to be incorporated by reference.

1. A combination of polynucleotides for amplification and detection of a S. aureus target nucleotide sequence, said combination comprising:
(a) a first polynucleotide primer comprising at least 7 consecutive nucleotides of the sequence as set forth in SEQ ID NO:1;
(b) a second polynucleotide primer comprising at least 7 consecutive nucleotides of a sequence complementary to SEQ ID NO:1; and
(c) a polynucleotide probe comprising at least 7 consecutive nucleotides of the sequence as set forth in SEQ ID NO:12, or the complement thereof.

2. The combination of polynucleotides according to claim 1, wherein said first polynucleotide primer comprises at least 7 consecutive nucleotides of the sequence as set forth in any one of SEQ ID NOs:2-11 and said second polynucleotide primer comprises at least 7 consecutive nucleotides of a sequence complementary to any one of SEQ ID NOs:2-11.

3. The combination of polynucleotides according to claim 1, wherein said target nucleotide sequence is a portion of the S. aureus femB gene sequence that is less than about 500 nucleotides in length and comprises at least 55 consecutive nucleotides of the sequence set forth in SEQ ID NO:12.

4. The combination of polynucleotides according to claim 1, wherein said first polynucleotide primer comprises at least 7 consecutive nucleotides of the sequence as set forth in SEQ ID NO:12 and said second polynucleotide primer comprises at least 7 consecutive nucleotides of a sequence complementary to SEQ ID NO:12.

5. The combination of polynucleotides according to claim 1, wherein said first polynucleotide primer comprises at least 7 consecutive nucleotides of the sequence as set forth in SEQ ID NO:14 or 20, said second polynucleotide primer comprises at least 7 consecutive nucleotides of the sequence as set forth in SEQ ID NO:15 and said polynucleotide probe comprises at least 7 consecutive nucleotides of the sequence as set forth in SEQ ID NO:17, or the complement thereof.

6. A method of detecting S. aureus in a sample, said method comprising:
(i) contacting a sample suspected of containing, or known to contain, a S. aureus target nucleotide sequence with a combination of polynucleotide primers capable of amplifying said target nucleotide sequence under conditions that permit amplification of said target nucleotide sequence, said polynucleotide primers comprising:
(a) a first polynucleotide primer comprising at least 7 consecutive nucleotides of the sequence as set forth in SEQ ID NO:1; and
(b) a second polynucleotide primer comprising at least 7 consecutive nucleotides of a sequence complementary to SEQ ID NO:1,
and said target nucleotide sequence being a portion of a S. aureus femB gene of less than about 500 nucleotides in length and comprising at least 55 consecutive nucleotides of the sequence set forth in SEQ ID NO:12, and
(ii) detecting any amplified target nucleotide sequence,
wherein detection of an amplified target nucleotide sequence indicates the presence of S. aureus in the sample.

7. The method according to claim 6, wherein said first polynucleotide primer comprises at least 7 consecutive nucleotides of the sequence as set forth in any one of SEQ ID NOs:2-11 and said second polynucleotide primer comprises at least 7 consecutive nucleotides of a sequence complementary to any one of SEQ ID NOs:2-11.

8. The method according to claim 6, wherein said first polynucleotide primer comprises at least 7 consecutive nucleotides of the sequence as set forth in SEQ ID NO:12 and said second polynucleotide primer comprises at least 7 consecutive nucleotides of a sequence complementary to SEQ ID NO:12.

9. The method according to claim 6, wherein step (ii) comprises detecting any amplified target nucleotide sequence by contacting said amplified target nucleotide sequences with a polynucleotide probe comprising at least 7 consecutive nucleotides of the sequence as set forth in SEQ ID NO:12, or the complement thereof.

10. The method according to claim 6, wherein steps (i) and (ii) are conducted simultaneously.

11. The method according to claim 6, further comprising a step to enrich the microbial content of the sample prior to step (a).

12. A method of detecting S. aureus in a sample, said method comprising the steps of:
(i) contacting a sample suspected of containing, or known to contain, a S. aureus target nucleotide sequence with a combination of polynucleotides under conditions that permit amplification of said target nucleotide sequence, wherein said combination of polynucleotides comprises:
(a) a first polynucleotide primer comprising at least 7 consecutive nucleotides of the sequence as set forth in SEQ ID NO:1;
(b) a second polynucleotide primer comprising at least 7 consecutive nucleotides of a sequence complementary to SEQ ID NO:1; and
(c) a polynucleotide probe comprising at least 7 consecutive nucleotides of the sequence as set forth in SEQ ID NO:12, or the complement thereof; and
(ii) detecting any amplified target nucleotide sequence,
wherein detection of an amplified target nucleotide sequence indicates the presence of S. aureus in the sample.

13. A kit for the detection of S. aureus in a sample, said kit comprising:
(a) a first polynucleotide primer comprising at least 7 consecutive nucleotides of the sequence as set forth in SEQ ID NO:1;
(b) a second polynucleotide primer comprising at least 7 consecutive nucleotides of a sequence complementary to SEQ ID NO:1; and
(c) a polynucleotide probe comprising at least 7 consecutive nucleotides of the sequence as set forth in SEQ ID NO:12, or the complement thereof.

14. The kit according to claim 13, wherein said first polynucleotide primer comprises at least 7 consecutive nucleotides of the sequence as set forth in any one of SEQ ID NOs:2-11 and said second polynucleotide primer comprises at least 7 consecutive nucleotides of a sequence complementary to any one of SEQ ID NOs:2-11.

15. The kit according to claim 13, wherein said first polynucleotide primer comprises at least 7 consecutive nucleotides of the sequence as set forth in SEQ ID NO:12 and said second polynucleotide primer comprises at least 7 consecutive nucleotides of a sequence complementary to SEQ ID NO:12.

16. The kit according to claim 13, wherein said first polynucleotide primer comprises at least 7 consecutive nucleotides of the sequence as set forth in SEQ ID NO:14 or 20, said second polynucleotide primer comprises at least 7 consecutive nucleotides of the sequence as set forth in SEQ ID NO:15 and said polynucleotide probe comprises at least 7 consecutive nucleotides of the sequence as set forth in SEQ ID NO:17, or the complement thereof.

17. The kit according to claim 13, further comprising one or more amplification reagents selected from the group of: buffers, salts, enzymes, enzyme co-factors, and nucleotides.

18. A pair of polynucleotide primers for amplification of a portion of a S. aureus femB gene sequence, said portion being less than about 500 nucleotides in length and comprising at least 55 consecutive nucleotides of the sequence set forth in SEQ ID NO:12, said pair of polynucleotide primers comprising:
(a) a first polynucleotide primer comprising at least 7 consecutive nucleotides of the sequence as set forth in SEQ ID NO:1; and
(b) a second polynucleotide primer comprising at least 7 consecutive nucleotides of a sequence complementary to SEQ ID NO:1.

19. The pair of polynucleotide primers according to claim 18, wherein said first polynucleotide primer comprises at least 7 consecutive nucleotides of the sequence as set forth in any one of SEQ ID NOs:2-11 and said second polynucleotide primer comprises at least 7 consecutive nucleotides of a sequence complementary to any one of SEQ ID NOs:2-11.

20. The pair of polynucleotide primers according to claim 18, wherein said first polynucleotide primer comprises at least 7 consecutive nucleotides of the sequence as set forth in SEQ ID NO:12 and said second polynucleotide primer comprises at least 7 consecutive nucleotides of a sequence complementary to SEQ ID NO:12.

21. The pair of polynucleotide primers according to claim 18, wherein said first polynucleotide primer comprises at least 7 consecutive nucleotides of the sequence as set forth in SEQ ID NO:14 or 20 and said second polynucleotide primer comprises at least 7 consecutive nucleotides of the sequence as set forth in SEQ ID NO:15.

22. An isolated S. aureus specific polynucleotide consisting essentially of:
(a) the sequence as set forth in SEQ ID NO:12, or a fragment of said sequence, or
(b) a sequence that is the complement of (a).

23. A polynucleotide primer of between 7 and 100 nucleotides in length for amplification of a portion of a S. aureus femB gene sequence, said polynucleotide primer comprising at least 7 consecutive nucleotides of the sequence as set forth in SEQ ID NO:12, or the complement thereof.

24. The polynucleotide primer according to claim 23, wherein said polynucleotide primer comprises at least 7 consecutive nucleotides of the sequence as set forth in any one of: SEQ ID NOs:14, 15, 17, 19 or 20.

25. A polynucleotide probe of between 7 and 100 nucleotides in length for detection of S. aureus nucleic acids, said polynucleotide probe comprising at least 7 consecutive nucleotides of the sequence as set forth in SEQ ID NO:12, or the complement thereof.

26. The polynucleotide probe according to claim 25, wherein said polynucleotide probe comprises at least 7 consecutive nucleotides of the sequence as set forth in SEQ ID NO:17, or the complement thereof.

27. The polynucleotide probe according to claim 25, wherein said polynucleotide probe comprises the sequence as set forth in SEQ ID NO:17, or the complement thereof.

28. The polynucleotide probe according to claim 25, wherein said polynucleotide probe further comprises a fluorophore, a quencher, or a combination thereof.
